SEQUENCE LISTING (8 sequences)

SEQ ID NO: 1 - DNA, 19 bp, synthetic construct
  misc_feature 1..19: note = PPAR-gamma-2 Forward primer
  source 1..19: mol_type = other DNA
gcgattccttcactgatac

SEQ ID NO: 2 - DNA, 20 bp, synthetic construct
  misc_feature 1..20: note = PPAR-gamma-2 Reverse primer
  source 1..20: mol_type = other DNA
gcattatgagacatccccac

SEQ ID NO: 3 - DNA, 20 bp, synthetic construct
  misc_feature 1..20: note = FABP4 (aP2) Forward primer
  source 1..20: mol_type = other DNA
aaccttagatgggggtgtcc

SEQ ID NO: 4 - DNA, 20 bp, synthetic construct
  misc_feature 1..20: note = FABP4 (aP2) Reverse primer
  source 1..20: mol_type = other DNA
atgcgaacttcagtccaggt

SEQ ID NO: 5 - DNA, 22 bp, synthetic construct
  misc_feature 1..22: note = GLUT-4 Forward primer
  source 1..22: mol_type = other DNA
ttccaacagataggctccgaag

SEQ ID NO: 6 - DNA, 20 bp, synthetic construct
  misc_feature 1..20: note = GLUT-4 Reverse primer
  source 1..20: mol_type = other DNA
aagcaccgcagagaacacag

SEQ ID NO: 7 - DNA, 19 bp, synthetic construct
  misc_feature 1..19: note = RPII Forward primer
  source 1..19: mol_type = other DNA
cttcacggtgctgggcatt

SEQ ID NO: 8 - DNA, 18 bp, synthetic construct
  misc_feature 1..18: note = RPII Reverse primer
  source 1..18: mol_type = other DNA
gtgcggctgcttccataa

(19) United States
(12) Patent Application Publication
(10) Pub. No.: US 2026/0176587 A1
(43) Pub. Date: Jun. 25, 2026

(54) ARTIFICIAL FAT TISSUE AND METHOD FOR PRODUCING SAME, METHOD FOR PRODUCING ARTIFICIAL SKIN AND AGENT FOR FAT CELL CULTURE

(71) Applicants: TOPPAN Holdings Inc., Tokyo (JP); The University of Osaka, Osaka (JP)

(72) Inventors: Michiya MATSUSAKI, Osaka (JP); Shiro KITANO, Tokyo (JP); Shinji IRIE, Tokyo (JP); Fiona LOUIS, Osaka (JP)

(73) Assignees: TOPPAN Holdings Inc., Tokyo (JP); The University of Osaka, Osaka (JP)

(21) Appl. No.: 19/467,373

(22) Filed: Feb. 2, 2026

Related U.S. Application Data
(63) Continuation-in-part of application No. 16/762,479, now abandoned.

(30) Foreign Application Priority Data
Nov. 10, 2017 (JP) 2017-217804

(51) Int. Cl. C12N 5/077

(52) U.S. Cl. CPC C12N 5/0653; C12N 2513/00; C12N 2533/90

(57) ABSTRACT

Disclosed is an artificial adipose tissue comprising a three-dimensional tissue comprising: a population of cells including an adipocyte; and a fragmented extracellular matrix.

### CROSS-REFERENCE TO RELATED APPLICATIONS

This application is a continuation-in-part of U.S. patent application Ser. No. 16/762,479, filed on May 7, 2020, which is a National Stage Entry of PCT/JP2018/041483 filed on Nov. 9, 2018, the disclosure of which is incorporated herein by reference in its entirety.

### REFERENCE TO AN ELECTRONIC SEQUENCE LISTING

The contents of the electronic sequence listing (FP18-1021-01US-TPN_SEQL.xml; Size: 11,292 bytes; and Date of Creation: Dec. 29, 2025) is herein incorporated by reference in its entirety.

### TECHNICAL FIELD

The present invention relates to an artificial adipose tissue and a method for producing the same, a method for producing an artificial skin, and an agent for adipocyte culture.

### BACKGROUND ART

In vitro long-term culture maintenance of mature adipocytes is one of important issues in the fields of, in particular, cosmetic/pharmaceutical assay or plastic surgery. The culturing period of conventional two-dimensional culture on a flat dish is short, and adipocytes are typically dedifferentiated after one week. A past report shows that uniocular mature adipocytes cannot be maintained over a period of one week or longer (Non Patent Literature 1). In addition, it has been difficult to change a culture medium because substantially more matured adipocytes tend to float over a culture dish due to their intracellular lipid droplets.

To provide a solution to these problems, three-dimensional cell culture technology has attracted attention. WAQAR HASSAN et al. have encapsulated and cultured cells in hydrogel having, as components, a PEG-based copolymer and hyaluronic acid (Non Patent Literature 2). Unfortunately, in this method, the cells are isolated and cultured, and as a result of which contact between the cells is physically difficult and their differentiation efficiency is thus poor.

DAQUINAG et al. have demonstrated that angiogenesis and lipogenesis can be simultaneously simulated using mouse 3T3-L1 preadipocytes and GFP-expressing mouse endothelial cells in a three-dimensionally structured culture system having magnetic nanoparticles as a platform. The coculture of adipocytes and endothelial cells are distinct by immunostaining, but fat vesicles at day 14 are very small and the maturity is low (Non Patent Literature 3).

Meanwhile, regarding an adipocyte-containing skin model, a report shows a skin model using cow collagen type I hydrogel (3 mg/mL) to structure a double layer of fibroblasts and adipose stem cells. However, the culture requires a very long period of from 56 days to 63 days so as to construct the skin model. Besides, the thickness is thinner than that of a human skin structure (Non Patent Literature 4).

In addition, there is a report on a skin model having three layers structured by fibroblasts, mesenchymal stem cells, and adipocytes resuspended in frozen human plasma hydrogel (1 mg/ml fibrinogen). The model construction, however, needs a long-term culturing process over 35 days. On top of that, a human skin structure was not successfully mimicked morphologically, and the skin was very thin (Non Patent Literature 5).

### CITATION LIST

### Non Patent Literature

Non Patent Literature 1: Toda S, Uchihashi K, Aoki S, et al., Organogenesis. 2009 5 (2): 50-56
Non Patent Literature 2: WAQAR HASSAN et al., STEM CELL RESEARCH & THERAPY 2013, 21; 4 (2): 32
Non Patent Literature 3: DAQUINAG, et al., TISSUE ENGINEERING. PART C, METHODS, 2013 May; 19 (5): 336-44.
Non Patent Literature 4: TROTTIER et al., STEM CELLS, 2008 October; 26 (10): 2713-23
Non Patent Literature 5: MONFORT et al., J Tissue Eng Regen Med., 2013 June; 7 (6): 479-90

### SUMMARY OF INVENTION

### Technical Problem

It is an object of one embodiment of the present invention to provide an artificial adipose tissue capable of being maintained for a long period of time and to provide a production method capable of producing the artificial adipose tissue in a short period. Further, it is an object of one embodiment of the present invention to provide an artificial skin including an artificial adipose tissue capable of being maintained for a long period of time and to provide a production method capable of producing the artificial skin in a short period. Furthermore, it is an object of one embodiment of the present invention to provide an agent for adipocyte culture and to provide a method for evaluating skin permeability of a compound by using the artificial skin.

### Solution to Problem

The present inventors have found that adipocytes, which are floating cells, and a fragmented extracellular matrix are co-cultured to form a three-dimensional tissue; the adipocytes are differentiated in a short period; and an artificial adipose tissue including the three-dimensional tissue formed can be maintained for a long period of time. Further, it has been found that use of the artificial adipose tissue allows for production of an artificial skin having a thickness close to the thickness of human skin.

Specifically, the present invention provides, for instance, the following [1] to [29].
[1] An artificial adipose tissue comprising a three-dimensional tissue comprising: a population of cells including an adipocyte; and a fragmented extracellular matrix.
[2] The artificial adipose tissue according to [1], wherein the extracellular matrix comprises collagen.
[3] The artificial adipose tissue according to [1] or [2], wherein content of the extracellular matrix is from 10 wt % to 30 wt % based on the artificial adipose tissue.
[4] The artificial adipose tissue according to any one of [1] to [3], wherein a thickness thereof is from 1 mm to 10 mm.
[5] The artificial adipose tissue according to any one of [1] to [4], wherein a contraction rate thereof during culturing is 20% or less.
[6] The artificial adipose tissue according to any one of [1] to [5], wherein an average length of the fragmented extracellular matrix is from 100 nm to 200 μm.
[7] The artificial adipose tissue according to any one of [1] to [6], wherein an average size of lipid droplets in the adipocyte is from 20 μm to 180 μm.
[7-A] The artificial adipose tissue according to any one of [1] to [7], wherein the pore area of the fragmented extracellular matrix is 10 μm² or more and 500 μm² or less.
[7-B] The artificial adipose tissue according to any one of [1] to [7-A], wherein the pore size of the fragmented extracellular matrix is 1 μm or more and 50 μm or less.
[7-C] The artificial adipose tissue according to any one of [1] to [7], wherein the fragmented extracellular matrix is fragmented through one or more cycles of freezing and thawing.
[7-D] The artificial adipose tissue according to [7-C], wherein the pore area of the fragmented extracellular matrix is 0.01 μm² or more and 1 μm² or less.
[7-E] The artificial adipose tissue according to [7-C] or [7-D], wherein the pore size of the fragmented extracellular matrix is 0.01 μm or more and 10 μm or less.
[7-F] The artificial adipose tissue according to any one of [1] to [7], wherein the fragmented extracellular matrix comprises a first population of fragmented extracellular matrix having a pore area of 10 μm² or more and 500 μm² or less; and a second population of fragmented extracellular matrix having a pore area of 0.01 μm² or more and 1 μm² or less.
[7-G] The artificial adipose tissue according to any one of [1] to [7] and [7-F], wherein the fragmented extracellular matrix comprises a first population of fragmented extracellular matrix having a pore size of 1 μm or more and 50 μm or less; and a second population of fragmented extracellular matrix having a pore size of 0.01 μm or more and 10 μm or less.
[7-H] The artificial adipose tissue according to any one of [1] to [7-G], wherein the fragmented extracellular matrix undergoes gelation from a sol state at 35.5° C.±2° C. and undergoes solation from a gel state at 4.5° C.±2° C.
[7-I] The artificial adipose tissue according to any one of [1] to [7-H], wherein the fluorescence intensity of the fragmented extracellular matrix at a wavelength of 510 nm is 50 a.u. or more and 300 a.u. or less, when measured with a spectrofluorometer using a fluorescent hydrophobic probe upon excitation at 380 nm.
[8] An artificial skin comprising a first layer and a second layer, wherein the first layer comprises the artificial adipose tissue according to any one of [1] to [7-I].
[9] The artificial skin according to [8], wherein the second layer comprises fibroblasts.
[10] The artificial skin according to [8] or [9], further comprising a third layer.
[11] The artificial skin according to [10], wherein the third layer comprises keratinocytes.
[12] The artificial skin according to any one of [8] to [11], wherein a thickness thereof is from 0.2 mm to 10 mm.
[13] A method for producing an artificial adipose tissue, comprising:
   (1) contacting a population of cells including an adipocyte with a fragmented extracellular matrix in an aqueous medium; and
   (2) culturing the cells contacted with the fragmented extracellular matrix.
[14] The production method according to [13], wherein a period of the culturing in step (2) is from 10 days to 30 days.
[15] The production method according to [13] or [14], further comprising, between step (1) and step (2), a step of subjecting the fragmented extracellular matrix and the cells to precipitate in the aqueous medium.
[16] The production method according to any one of [13] to [15], wherein the extracellular matrix comprises collagen.
[17] The production method according to any one of [13] to [16], wherein an average length of the fragmented extracellular matrix is from 100 nm to 200 μm.
[17-A] The production method according to any one of [13] to [17], wherein the pore area of the fragmented extracellular matrix is 10 μm² or more and 500 μm² or less.
[17-B] The production method according to any one of [13] to [17-A], wherein the pore size of the fragmented extracellular matrix is 1 μm or more and 50 μm or less.
[17-C] The production method according to any one of [13] to [17], wherein the fragmented extracellular matrix is fragmented through one or more cycles of freezing and thawing.
[17-D] The production method according to [17-C], wherein the pore area of the fragmented extracellular matrix is 0.01 μm² or more and 1 μm² or less.
[17-E] The production method according to [17-D], wherein the pore size of the fragmented extracellular matrix is 0.01 μm or more and 10 μm or less.
[17-F] The production method according to any one of [13] to [17], wherein the fragmented extracellular matrix comprises a first population of fragmented extracellular matrix having a pore area of 10 μm² or more and 500 μm² or less; and a second population of fragmented extracellular matrix having a pore area of 0.01 μm² or more and 1 μm² or less.
[17-G] The production method according to any one of [13] to [17-F], wherein the fragmented extracellular matrix comprises a first population of fragmented extracellular matrix having a pore size of 1 μm or more and 50 μm or less; and a second population of fragmented extracellular matrix having a pore size of 0.01 μm or more and 10 μm or less.
[17-H] The production method according to any one of [13] to [17-G], wherein the fragmented extracellular matrix undergoes gelation from a sol state at 35.5° C.±2° C. and undergoes solation from a gel state at 4.5° C.±2° C.
[17-I] The production method according to any one of [13] to [17-H], wherein the NaCl concentration of the aqueous medium in step (1) is 13.0 mM or more and 1400 mM or less.
[17-J] The production method according to any one of [13] to [17-I], wherein the fluorescence intensity of the fragmented extracellular matrix at a wavelength of 510 nm is 50 a.u. or more and 300 a.u. or less, when measured with a spectrofluorometer using a fluorescent hydrophobic probe upon excitation at 380 nm.
[18] A method for producing an artificial skin, comprising:
   forming a first layer, comprising (1) contacting first a population of cells including an adipocyte with a first fragmented extracellular matrix in an aqueous medium, and
(2) culturing the first cells contacted with the first fragmented extracellular matrix; and
   forming a second layer, comprising (3) further contacting the first layer with second population of cells and culturing the second population of cells.
[19] The method for producing an artificial skin according to [18], wherein an average length of the first fragmented extracellular matrix is from 100 nm to 200 μm.
[19-A] The production method according to [18] or [19], wherein the pore area of the first fragmented extracellular matrix is 10 μm² or more and 500 μm² or less.
[19-B] The production method according to any one of [18] to [19-I], wherein the pore size of the first fragmented extracellular matrix is 1 μm or more and 50 μm or less.
[19-C] The production method according to any one of [18] to [19], wherein the first fragmented extracellular matrix is fragmented through one or more cycles of freezing and thawing.
[19-D] The production method according to [19-C], wherein the pore area of the first fragmented extracellular matrix is 0.01 μm² or more and 1 μm² or less.
[19-E] The production method according to [19-D], wherein the pore size of the first fragmented extracellular matrix is 0.01 μm or more and 10 μm or less.
[19-F] The production method according to [18] or [19], wherein the first fragmented extracellular matrix comprises a first population of the first fragmented extracellular matrix having a pore area of 10 μm² or more and 500 μm² or less; and a second population of the first fragmented extracellular matrix having a pore area of 0.01 μm² or more and 1 μm² or less.
[19-G] The production method according to any one of [18] to [19-F], wherein the first fragmented extracellular matrix comprises a first population of the first fragmented extracellular matrix having a pore size of 1 μm or more and 50 μm or less; and a second population of the first fragmented extracellular matrix having a pore size of 0.01 μm or more and 10 μm or less.
[19-H] The production method according to any one of [18] to [19-G], wherein the first fragmented extracellular matrix undergoes gelation from a sol state at 35.5° C.±2° C. and undergoes solation from a gel state at 4.5° C.±2° C.
[19-I] The production method according to any one of [18] to [19-H], wherein the NaCl concentration of the aqueous medium in step (1) is 13.0 mM or more and 1400 mM or less.
[19-J] The production method according to any one of [18] to [19-I], wherein the fluorescence intensity of the first fragmented extracellular matrix at a wavelength of 510 nm is 50 a.u. or more and 300 a.u. or less, when measured with a spectrofluorometer using a fluorescent hydrophobic probe upon excitation at 380 nm.
[20] The production method according to [18] or [19], wherein a period of the culturing in step (2) is from 10 days to 60 days.
[21] The method for producing an artificial skin according to any one of [18] to [20], wherein step (3) is a step comprising contacting the second population of cells with a second fragmented extracellular matrix on the first layer in an aqueous medium and culturing the second cells. [21-A] The production method according to [21], wherein the pore area of the second fragmented extracellular matrix is 10 μm² or more and 500 μm² or less.
[21-B] The production method according to [21] or [21-A], wherein the pore size of the second fragmented extracellular matrix is 1 μm or more and 50 μm or less.
[21-C] The production method according to [21], wherein the second fragmented extracellular matrix is fragmented through one or more cycles of freezing and thawing.
[21-D] The production method according to [21-C], wherein the pore area of the second fragmented extracellular matrix is 0.01 μm² or more and 1 μm² or less.
[21-E] The production method according to [21-D], wherein the pore size of the second fragmented extracellular matrix is 0.01 μm or more and 10 μm or less.
[21-F] The production method according to [21], wherein the second fragmented extracellular matrix comprises a first population of the second fragmented extracellular matrix having a pore area of 10 μm² or more and 500 μm² or less; and a second population of the second fragmented extracellular matrix having a pore area of 0.01 μm² or more and 1 μm² or less.
[21-G] The production method according to [21], wherein the second fragmented extracellular matrix comprises a first population of the second fragmented extracellular matrix having a pore size of 1 μm or more and 50 μm or less; and a second population of the second fragmented extracellular matrix having a pore size of 0.01 μm or more and 10 μm or less.
[21-H] The production method according to any one of [21] to [21-G], wherein the second fragmented extracellular matrix undergoes gelation from a sol state at 35.5° C.±2° C. and undergoes solation from a gel state at 4.5° C.±2° C.
[21-I] The production method according to any one of [21] to [21-H], wherein the NaCl concentration of the aqueous medium in step (3) is 13.0 mM or more and 1400 mM or less.
[21-J] The production method according to any one of [21] to [21-I], wherein the fluorescence intensity of the second fragmented extracellular matrix at a wavelength of 510 nm is 50 a.u. or more and 300 a.u. or less, when measured with a spectrofluorometer using a fluorescent hydrophobic probe upon excitation at 380 nm.
[22] The method for producing an artificial skin according to [21], wherein the second extracellular matrix comprises collagen.
[23] The method for producing an artificial skin according to any one of [18] to [22], wherein the second population of cells comprise fibroblasts.
[24] The method for producing an artificial skin according to any one of [18] to [23], further comprising
   forming a third layer, comprising further contacting the second layer with third population of cells and culturing the third population of cells.
[25] The method for producing an artificial skin according to [24], wherein the third population of cells comprise keratinocytes.
[26] An agent for culturing an adipocyte, comprising a fragmented extracellular matrix.
[27] The agent for culturing an adipocyte according to [26], wherein the fragmented extracellular matrix is collagen having an average length of from 100 nm to 200 μm.
[28] The agent for culturing an adipocyte according to [26] or [27], which is a differentiation promoter.
[29] A method for evaluating skin permeability of a compound, comprising:
   providing the artificial skin according to any one of [8] to [12];
   contacting the artificial skin with a test compound;
   measuring skin permeability of the test compound using the artificial skin contacted with the test compound; and
   comparing between the skin permeability of the test compound and a reference value.

### Advantageous Effects of Invention

According to the present invention, there can be provided an artificial adipose tissue capable of being maintained for a long period of time and to provide a production method capable of producing the artificial adipose tissue in a short period. Further, according to the present invention, there can be provided an artificial skin including an artificial adipose tissue capable of being maintained for a long period of time and to provide a production method capable of producing the artificial skin in a short period. Furthermore, according to the present invention, there can be provided an agent for adipocyte culture and to provide a method for evaluating skin permeability of a compound by using the artificial skin.

Moreover, according to the present invention, there can be provided an artificial skin having a thickness close to the thickness of human skin and a production method thereof.

### BRIEF DESCRIPTION OF DRAWINGS

FIG. 1 is a schematic diagram illustrating the steps of producing an artificial adipose tissue including a three-dimensional tissue containing a fragmented collagen and mature adipocytes or adipose stem cells.

FIG. 2 is photographs showing the results of evaluating an artificial adipose tissue at day 14 of culturing in Example 1. Photograph A shows hematoxylin/eosin (HE) staining results. The arrow indicates a site where fragmented collagen is present. Each dashed line indicates a mature adipocyte. Photograph B shows the results of Nile red lipid staining. White portions indicate nuclei. Photograph C shows the results using a Live/Dead kit. Grey indicates living cells and white indicates dead cells.

FIG. 3 is photographs showing the anti-perilipin antibody immunostaining results of an artificial adipose tissue in Example 1 and an artificial adipose tissue produced by conventional two-dimensional culture. White portions indicate nuclei. The “In 3D” photograph shows the results of the artificial adipose tissue in Example 1 and the “In 2D” photograph shows the results of the artificial adipose tissue produced by conventional two-dimensional culture.

FIG. 4 is a graph showing the results of comparing the fat vesicle size between the artificial adipose tissue in Example 1 and the artificial adipose tissue produced by conventional two-dimensional culture at day 7 of culturing and day 14 (*p<0.001). In the graph, the “3D” indicates the results of the artificial adipose tissue in Example 1 and the “2D” indicates the results of the artificial adipose tissue produced by conventional two-dimensional culture.

FIG. 5 is photographs showing the HE staining results of an artificial adipose tissue at day 17 of differentiation in Example 2. The upper photograph is a photographic image in which part of the photograph below has been enlarged. Each dashed line indicates a mature adipocyte. The arrow indicates a site where fragmented collagen is present.

FIG. 6 is electron micrographs of artificial adipose tissues produced by conventional two-dimensional culture at day 37 and day 72 of differentiation. The upper photograph shows an artificial adipose tissue at day 37 of differentiation, and the lower photograph shows an artificial adipose tissue at day 72 of differentiation. The arrow indicates a mature adipocyte.

FIG. 7 is photographs showing the anti-perilipin antibody immunostaining results of artificial adipose tissues in Example 2 and artificial adipose tissues produced by conventional two-dimensional culture. White portions indicate nuclei. The “In 3D” photographs show the results of the artificial adipose tissues in Example 2 and the “In 2D” photographs show the results of the artificial adipose tissues produced by conventional two-dimensional culture.

FIG. 8 is a graph showing the results of comparing the fat vesicle size between the artificial adipose tissues in Example 2 and the artificial adipose tissues produced by conventional two-dimensional culture at day 7 of culturing and day 14 (*p<0.001). In the graph, the “3D” indicates the results of the artificial adipose tissues in Example 2 and the “2D” indicates the results of the artificial adipose tissues produced by conventional two-dimensional culture.

FIG. 9 is a schematic diagram illustrating the steps of producing an artificial skin including: a first layer including an artificial adipose tissue containing a fragmented collagen and mature adipocytes;

a second layer containing fibroblasts; and a third layer containing keratinocytes.

FIG. 10 is photographs showing the HE staining results of an artificial skin (left) in Example 3, which was produced, using 1×10⁶ adipocytes and 10 mg fragmented collagen, at day 9 of culturing (keratinocyte day 7 of differentiation) and a mouse subcutaneous tissue-containing skin (right).

FIG. 11 is photographs showing the HE staining results of an artificial skin in Example 3, which was produced, using 1×10⁶ adipocytes and 10 mg fragmented collagen, at day 9 of culturing (keratinocyte day 7 of differentiation). In photograph A, a keratinocyte-containing layer is enlarged. In photograph B, a fibroblast-containing layer is enlarged. In photograph C, an adipocyte-containing layer is enlarged.

FIG. 12 is photographs showing the HE staining results of an artificial skin in Example 3, which was produced, using 5×10⁵ adipocytes and 10 mg fragmented collagen, at day 9 of culturing (keratinocyte day 7 of differentiation). In photograph A, a keratinocyte-containing layer is enlarged. In photograph B, a fibroblast-containing layer is enlarged. In photograph C, an adipocyte-containing layer is enlarged.

FIG. 13 is photographs showing the HE staining results of an artificial skin in Example 3, which was produced, using 1×10⁶ adipocytes and 15 mg fragmented collagen, at day 9 of culturing (keratinocyte day 7 of differentiation). In photograph A, a keratinocyte-containing layer is enlarged. In photograph B, a fibroblast-containing layer is enlarged. In photograph C, an adipocyte-containing layer is enlarged.

FIG. 14 is a graph showing the relationship between the keratinocyte differentiation time course and the artificial skin thicknesses of artificial skins having a three-layer structure produced in Example 3. The closed squares indicate the results of the artificial skin produced using 5×10⁵ adipocytes and 10 mg fragmented collagen in Example 3. The closed circles indicate the results of the artificial skin produced using 1×10⁶ adipocytes and 10 mg fragmented collagen in Example 3. The open circles indicate the results of the artificial skin produced using 1×10⁶ adipocytes and 15 mg fragmented collagen in Example 3.

FIG. 15 shows the results of evaluating, using RT-qPCR, the levels of expression of adipogenesis genes PPARγ2, FABP4, and GLUT4 until day 21 of differentiation between a three-dimensional human adipose stem cell-containing tissue (3D) and an artificial adipose tissue (2D) produced by conventional two-dimensional culture, which levels are normalized by the level of expression of a housekeeping gene RPII. The “*” indicates a significant difference with respect to the two-dimensional tissue (*p<0.05, ** p<0.01, and *** p<0.001).

FIG. 16 shows SEM images comparing Matrigel, collagen micro fiber (CMF), and collagen nano fiber (CNF).

FIG. 17 shows the analysis of fiber diameter using Phase Contrast images for CMF.

FIG. 18 shows the analysis of fiber diameter using TEM images for CMF.

FIG. 19 is a histogram showing the fiber diameter distribution of CMF.

FIG. 20 shows the analysis of fiber diameter using Phase Contrast images for CNF.

FIG. 21 shows the analysis of fiber diameter using TEM images for CNF.

FIG. 22 is a histogram showing the fiber diameter distribution of CNF.

FIG. 23 is a graph showing the viscoelastic properties (G′, G″) of the CMF sol at room temperature.

FIG. 24 is a graph showing the viscoelastic properties (G′, G″) of the CMF gel at 37° C.

FIG. 25 is a graph showing the viscoelastic properties (G′, G″) of the CNF sol at 4° C.

FIG. 26 is a graph showing the viscoelastic properties (G′, G″) of the CNF gel at 37° C.

FIG. 27 is a graph showing the viscoelastic properties (G′, G″) of Matrigel at 37° C.

FIG. 28 is a graph showing the viscoelastic properties (G′, G″) of Nippi collagen at 37° C.

FIG. 29 is a graph showing the results of comparing the elastic modulus of CMF and CNF gels at various concentrations, as well as Matrigel. The “*” indicates a significant difference (*p<0.05, ** p<0.005, and *** p<0.001).

FIG. 30 is a graph showing the fluorescence spectra of CMF gel, CNF gel, and PBS using a hydrophobic probe (ANS-Na).

FIG. 31 is a fluorescence micrograph of a Nippi collagen gel.

FIG. 32 is a fluorescence micrograph of a CMF gel.

FIG. 33 is a fluorescence micrograph of a CNF gel.

FIG. 34 is a graph showing the quantitative analysis of fluorescence intensity per unit area for the gels shown in FIGS. 31-33.

FIG. 35 is a graph showing the effect of salt concentration (0 mM vs. 137 mM NaCl) on the sol-gel transition of CNF, measured by transmittance.

FIG. 36 is a photograph showing the effect of collagen concentration (0.1 wt % vs. 1.0 wt %) on the gelation of CNF at 37° C.

FIG. 37 is a series of photographs illustrating the effect of pH (acidic conditions) on the sol-gel transition behavior of CNF.

FIG. 38 is a photograph showing the state of CMF prepared by a method of Example 12.

FIG. 39 is a photograph showing the state of CMF prepared by the method of Example 12 after incubation at 4° C.

FIG. 40 is a photograph showing the state of CMF prepared by the method of Example 12 after heating to 37° C.

FIG. 41 is a series of photographs showing the results of the sol-gel transition of CNF prepared with accurately adjusted salt concentrations (after dialysis).

FIG. 42 is a graph showing the storage modulus (G′) of 1% CNF at various salt concentrations.

FIG. 43 is a graph showing the loss modulus (G″) of 1% CNF at various salt concentrations.

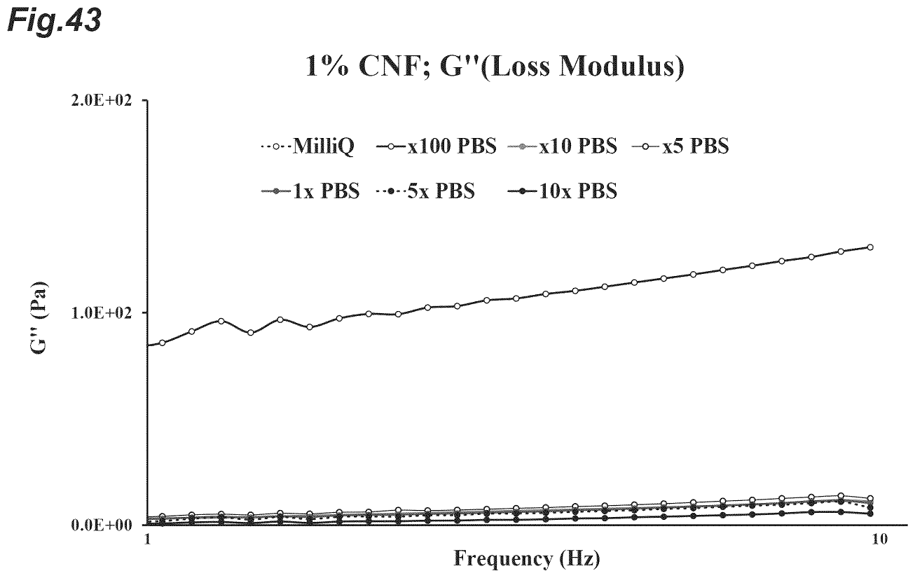

### DESCRIPTION OF EMBODIMENTS

### (Artificial Adipose Tissue)

An artificial adipose tissue according to an embodiment of the invention includes a three-dimensional tissue including a population of cells containing an adipocyte and a fragmented extracellular matrix. At least part of the cells adhere to the fragmented extracellular matrix. Differentiation of the artificial adipose tissue according to this embodiment is promoted more than that of an artificial adipose tissue produced by conventional two-dimensional culture on a flat dish, and the artificial adipose tissue according to this embodiment can be maintained over a long period of time.

The artificial adipose tissue according to this embodiment is an artificially prepared adipose tissue and does not include an adipose tissue simply isolated from a biological tissue.

The “three-dimensional tissue” is a cell assembly in which cells are three-dimensionally arranged via an extracellular matrix such as collagen and means an assembly artificially prepared by cell culture. The shape of the three-dimensional tissue is not particularly limited. Examples include a sheet shape, a spherical shape, an ellipsoid shape, and a rectangular parallelepiped shape. Here, the artificial adipose tissue of this embodiment, which includes a three-dimensional tissue containing a fragmented extracellular matrix, is distinguishable, depending on the presence or absence of the fragmented extracellular matrix, from a biological tissue or a three-dimensional tissue produced by other methods without using the fragmented extracellular matrix.

Examples of the “adipocyte” in this embodiment include not only a differentiated cell such as a mature adipocyte but also an undifferentiated cell such as an adipose stem cell. As the adipocyte(s), it is possible to use a cell(s) collected from, for instance, a subcutaneous adipose tissue or an epicardium-derived adipose tissue. Also, differentiation of the collected cell(s) may be induced and then used. The adipocyte(s) is not particularly limited. It is preferable that when an adipose tissue constructed from adipocytes is utilized while finally seen as an in vivo tissue at a specific site, adipocytes derived from a tissue corresponding to the tissue at the site are used. Examples of animal species of the adipocyte include a human, a mouse, a rat, or a pig. Preferable examples of the adipocyte(s) include a mature adipocyte(s) or an adipose stem cell(s).

The “extracellular matrix” means an extracellularly existing material in an organism. Specific examples include collagen, elastin, proteoglycan, fibronectin, or hyaluronic acid. The extracellular matrix in this embodiment is preferably a material extracellularly present in an animal, namely, an extracellular matrix in an animal, more preferably contains collagen or elastin, is still more preferably collagen or elastin, and is particularly preferably collagen.

Examples of the collagen include fibrillar collagen or non-fibrillar collagen. The fibrillar collagen means collagen that is a main component of collagen fiber. Specific examples of the fibrillar collagen include type I collagen, type II collagen, or type III collagen. Examples of the non-fibrillar collagen include type IV collagen.

Conventional three-dimensional tissue bodies have had a low concentration of extracellular matrix such as collagen and a high cell density. Thus, there have been problems that the three-dimensional tissue bodies are contracted by cell contraction force during or after culturing and the three-dimensional tissue bodies are readily lysed by an enzyme(s) produced by cells during or after culturing. In addition, although porous high-density collagen, for instance, is commercially available, it cannot cause collagen and cells to evenly adhere and it is thus hard to recover cells to evaluate the cell characteristics. An adipose tissue including a three-dimensional tissue according to this embodiment has a higher extracellular matrix content than those of conventional three-dimensional tissue bodies. Accordingly, the adipose tissue is unlikely to contract and is thus stable.

The extracellular matrix content in the three-dimensional tissue may be from 0.01 to 90 wt %, preferably from 10 to 90 wt %, preferably from 1 to 50 wt %, more preferably from 10 to 30 wt %, and particularly preferably from 20 to 30 wt % based on the above three-dimensional tissue. As used herein, the “extracellular matrix in a three-dimensional tissue” means an extracellular matrix as a component of the three-dimensional tissue, and may be an endogenous or exogenous extracellular matrix. Also, examples of the “extracellular matrix in a three-dimensional tissue” include a fragmented extracellular matrix described later. Specifically, if the three-dimensional tissue includes an endogenous extracellular matrix, the concentration of extracellular matrix as a component of the above three-dimensional tissue means a sum of the concentrations of the endogenous extracellular matrix and the fragmented extracellular matrix. The above extracellular matrix concentration may be calculated from the volume of the resulting three-dimensional tissue and the mass of the decellularized three-dimensional tissue.

The “endogenous extracellular matrix” means an extracellular matrix produced by extracellular matrix-producing cells. The “endogenous collagen” means collagen produced by collagen-producing cells as a component of the three-dimensional tissue. The endogenous collagen may be fibrillar collagen and/or non-fibrillar collagen.

The “exogenous extracellular matrix” means an extracellular matrix that is externally supplied. An artificial adipose tissue according to this embodiment includes a three-dimensional tissue, which includes a fragmented extracellular matrix. An original animal species of the exogenous extracellular matrix may be the same as of exogenous extracellular matrix or may be different therefrom. Examples of the original animal species include a human, a pig, and a cow. In addition, the exogenous extracellular matrix may be a synthetic extracellular matrix. The fragmented extracellular matrix is an exogenous extracellular matrix. In the case of collagen, the extracellular matrix is called “exogenous collagen” and means collagen that is externally supplied. Specific examples include fibrillar collagen or non-fibrillar collagen. It is preferable that the exogenous collagen be fibrillar collagen. Examples of the fibrillar collagen include type I collagen, type II collagen, and type III collagen. Preferred is type I collagen. Commercially available collagen may be used as the above fibrillar collagen. Specific examples include lyophilized, pig skin-derived type I collagen manufactured by NH Foods Ltd. Examples of the non-fibrillar exogenous collagen include type IV collagen.

An original animal species of the exogenous extracellular matrix may be different from that of the cells. When the cells contain extracellular matrix-producing cells, an original animal species of the exogenous extracellular matrix may be different from that of the extracellular matrix-producing cells. That is, the exogenous extracellular matrix may be a heterologous extracellular matrix.

The above three-dimensional tissue includes a fragmented extracellular matrix. The “fragmented extracellular matrix” means those obtained by fragmenting an extracellular matrix such as collagen. Regarding an extracellular matrix as a source of the fragmented extracellular matrix, one kind may be used, or multiple kinds of extracellular matrix may be used in combination. Conventionally, an extracellular matrix such as collagen has been dissolved in, for instance, an acidic aqueous solution. However, the concentration is from about 0.1 to 0.3 wt %. Accordingly, a large amount of extracellular matrix cannot be dissolved. Consequently, it is difficult to increase the amount of extracellular matrix, such as collagen, in the three-dimensional tissue by conventional methods. A fragmented extracellular matrix in this embodiment is almost immiscible in water. However, when dispersed in the below-described aqueous medium, the fragmented extracellular matrix is likely to be contacted with the population of cells, which include an adipocyte, in the aqueous medium, thereby possibly promoting formation of the three-dimensional tissue. The average length of the fragmented extracellular matrix is preferably from 100 nm to 400 μm, from 100 nm to 300 μm, or from 100 nm to 200 μm, more preferably from 22 μm to 200 μm, and still more preferably from 100 μm to 200 μm. The average diameter of the fragmented extracellular matrix is preferably from 50 nm to 30 μm, more preferably from 4 μm to 30 μm, and still more preferably from 20 μm to 30 μm.

When the extracellular matrix is collagen, the fragmented extracellular matrix is also called “fragmented collagen”. The “fragmented collagen” is those produced by fragmenting collagen such as fibrillar collagen and means those in which a triple helix structure is kept. The average length of the fragmented collagen is preferably from 100 nm to 400 μm, from 100 nm to 300 μm, or from 100 nm to 200 μm, more preferably from 22 μm to 200 μm, and still more preferably from 100 μm to 200 μm. The average diameter of the fragmented collagen is preferably from 50 nm to 30 μm, more preferably from 4 μm to 30 μm, and still more preferably from 20 μm to 30 μm.

A process for fragmenting an extracellular matrix such as collagen is not particularly limited. For instance, the extracellular matrix may be fragmented by using a homogenizer such as an ultrasonic homogenizer, a stirring homogenizer, or a high-pressure homogenizer. When the stirring homogenizer is used, an extracellular matrix, as it is, may be homogenized or may be homogenized in an aqueous medium such as saline. In addition, the duration and frequency of the homogenization may be adjusted to produce a fragmented extracellular matrix in mm or nm size. Alternatively, the fragmented extracellular matrix can be obtained by fragmenting through one or more cycles of freezing and thawing. The fragmented extracellular matrix may be fragmented through one or more cycles of freezing and thawing.

When homogenizing the extracellular matrix in an aqueous medium, pre-treatments such as neutralization, heating, and freeze-drying may be performed before homogenization, but it is preferable to perform homogenization without these pre-treatments.

The fragmented extracellular matrix component may be naturally derived. The fragmented extracellular matrix component that is naturally derived is a fragmented product of a natural extracellular matrix component, and components obtained by modifying the structure of a natural extracellular matrix molecule, without harsh chemical treatment. As used herein, “harsh chemical treatment” means a treatment that results in the substantial loss of the native structure and/or biological activity of the natural extracellular matrix. Examples of the harsh chemical treatment, which is not a means for obtaining the disclosed extracellular matrix component, include hydrolysis with alkali treatment.

The naturally derived fragmented extracellular matrix component may be one that has been subjected to a mild chemical treatment. As used herein, “mild chemical treatment” means a chemical treatment other than a harsh chemical treatment, that results in the substantially retains the native structure and/or biological activity of the natural extracellular matrix. Examples of such mild chemical treatments include, but are not limited to, enzymatic treatment (such as with pepsin).

The fragmented extracellular matrix may be the fragmented extracellular matrix having an average diameter of nano-order (1000 nm or less) (a nanofiber (NF)) or the fragmented extracellular matrix having an average diameter in the micro-order (more than 1000 nm) (a microfiber (MF)). The fragmented extracellular matrix is preferably the fragmented collagen. The fragmented extracellular matrix of this embodiment may be a collagen nanofiber (CNF), a collagen microfiber (CMF), or a mixture thereof.

The average diameter of the nanofibers may be, for example, 20 nm or more and 1000 nm or less, 20 nm or more and 500 nm or less, 20 nm or more and 400 nm or less, 20 nm or more and 300 nm or less, 20 nm or more and 200 nm or less, 20 nm or more and 190 nm or less, 20 nm or more and 180 nm or less, 20 nm or more and 170 nm or less, 20 nm or more and 160 nm or less, 20 nm or more and 150 nm or less, 20 nm or more and 140 nm or less, 20 nm or more and 130 nm or less, or 20 nm or more and 120 nm or less. The average diameter of the microfibers may be, for example, more than 1 μm and 30 μm or less, more than 1 μm and 25 μm or less, more than 1 μm and 20 μm or less, more than 1 μm and 15 μm or less, more than 1 μm and 10 μm or less, more than 1 μm and 9 μm or less, 1.5 μm or more and 8.5 μm or less, or 2 μm or more and 8.5 μm or less.

The pore area of the fragmented extracellular matrix can be appropriately adjusted by the type, average length, and average diameter of the fragmented extracellular matrix. The pore area of the fragmented extracellular matrix may be, for example, 10 μm² or more and 500 μm² or less, 20 μm² or more and 400 μm² or less, 30 μm² or more and 300 μm² or less, 40 μm² or more and 200 μm² or less, or 50 μm² or more and 100 μm² or less. Alternatively, the pore area of the fragmented extracellular matrix may be, for example, 0.01 μm² or more and 1 μm² or less, 0.02 μm² or more and 0.8 μm² or less, 0.05 μm² or more and 0.7 μm² or less, 0.08 μm² or more and 0.6 μm² or less, or 0.1 μm² or more and 0.5 μm² or less.

The pore size of the fragmented extracellular matrix can be appropriately adjusted by the type, average length, and average diameter of the fragmented extracellular matrix. The pore size of the fragmented extracellular matrix may be, for example, 1 μm or more and 50 μm or less, 2 μm or more and 40 μm or less, 3 μm or more and 30 μm or less, or 5 μm or more and 25 μm or less. Alternatively, the pore size of the fragmented extracellular matrix may be, for example, 0.01 μm or more and 10 μm or less, 0.02 μm or more and 5 μm or less, 0.03 μm or more and 2 μm or less, or 0.5 μm or more and 1.5 μm or less.

The diameter, the length, pore area and pore size of the fragmented extracellular matrix may be calculated by analyzing individual fragmented extracellular matrix by electron microscopy.

The elastic modulus of the fragmented extracellular matrix can be appropriately adjusted by the type, average length, and average diameter of the fragmented extracellular matrix. The elastic modulus of the fragmented extracellular matrix may be, for example, 10 kPa or more and 100 kPa or less, 20 kPa or more and 100 kPa or less, 30 kPa or more and 100 kPa or less, 40 kPa or more and 100 kPa or less, or 50 kPa or more and 100 kPa or less.

The fragmented extracellular matrix may have a storage modulus

(G′) at 37° C. in a frequency range of 0.1 to 1 Hz of, for example, 15 Pa or more and 500 Pa or less, 20 Pa or more and 500 Pa or less, 25 Pa or more and 500 Pa or less, 30 Pa or more and 500 Pa or less, 35 Pa or more and 500 Pa or less, 40 Pa or more and 500 Pa or less, 45 Pa or more and 500 Pa or less, 50 Pa or more and 500 Pa or less, 55 Pa or more and 500

Pa or less, 60 Pa or more and 500 Pa or less, 65 Pa or more and, 70 Pa or more and 500 Pa or less, 75 Pa or more and 500 Pa or less, 80 Pa or more and 500 Pa or less, 85 Pa or more and 500 Pa or less, 90 Pa or more and 500 Pa or less, 95 Pa or more and 500 Pa or less, or 100 Pa or more and 500 Pa or less.

The fragmented extracellular matrix may have a loss modulus (G″) at 37° C. in a frequency range of 0.1 to 1 Hz of, for example, 10 Pa or more and 500 Pa or less, 15 Pa or more and 500 Pa or less, 20 Pa or more and 500 Pa or less, 25 Pa or more and 500 Pa or less, 30 Pa or more and 500 Pa or less, 35 Pa or more and 500 Pa or less, 40 Pa or more and 500 Pa or less, 45 Pa or more and 500 Pa or less, 50 Pa or more and 500 Pa or less.

The fragmented extracellular matrix, under the condition that the salt concentration is 13 mM or more and 1400 mM or less, may be in a sol state. Specifically, under the condition that the salt concentration is, 13.7 mM or more and 1400 mM or less, 25.0 mM or more and 1400 mM or less, 27.4 mM or more and 1400 mM or less, 125.0 mM or more and 1400 mM or less, 137.0 mM or more and 1400 mM or less, or 625.0 mM or more and 1400 mM or less, at 4° C., the loss modulus (G″) measured in a frequency range of 1.0 Hz to 10.0 Hz may be greater than the storage modulus (G′). In that case, the ratio of the loss modulus (G″) to the storage modulus (G′) may be, for example, 2 or more, or 3 or more. Also, the storage modulus (G′) and the loss modulus (G″) may be, for example, below 100 Pa, 90 Pa, 80 Pa, 70 Pa, 60 Pa, 50 Pa, 40 Pa, 30 Pa, or 20 Pa.

In the present specification, the elastic modulus, storage modulus, and loss modulus are values measured by the methods described in the Examples below.

The fluorescence intensity of the fragmented extracellular matrix at a wavelength of 510 nm may be, for example, 50 a.u. or more and 300 a.u. or less, 60 a.u. or more and 300 a.u. or less, 70 a.u. or more and 300 a.u. or less, 80 a.u. or more and 300 a.u. or less, 90 a.u. or more and 300 a.u. or less, 100 a.u. or more and 300 a.u. or less, 110 a.u. or more and 300 a.u. or less, 120 a.u. or more and 300 a.u. or less, 130 a.u. or more and 300 a.u. or less, 140 a.u. or more and 300 a.u. or less, or 150 a.u. or more and 300 a.u. or less, when measured with a spectrofluorometer using a fluorescent hydrophobic probe (e.g., 8-anilino-1-naphthalenesulfonate; ANS-Na) upon excitation at 380 nm.

The fluorescence intensity per unit area of the fragmented extracellular matrix in a wavelength range of 500 nm to 550 nm may be, for example, 30 a.u./μm² or more and 150 a.u./μm² or less, 40 a.u./μm² or more and 150 a.u./μm² or less, 50 a.u./μm² or more and 150 a.u./μm² or less, 60 a.u./μm² or more and 150 a.u./μm² or less, 70 a.u./μm² or more and 150 a.u./μm² or less, or 80 a.u./μm² or more and 150 a.u./μm² or less, when observed with a fluorescence microscope using a fluorescent hydrophobic probe (e.g., ANS-Na) upon excitation at 405 nm.

The fragmented extracellular matrix is in a sol state at low temperature, and in a gel state at high temperature. For example, the fragmented extracellular matrix may undergo the progression of solation in a temperature range of at least 0° C. or more and 15° C. or less, and undergo the progression of gelation in a temperature range of at least 25° C. or more. In the case that the aqueous medium is phosphate-buffered saline, the fragmented extracellular matrix may be in a gel state at 37° C. and in a sol state at 4° C. Being in a sol state and being in a gel state can be confirmed by visual observation.

The temperature at which the fragmented extracellular matrix undergoes gelation from a sol state (gel transition temperature) may be, for example, 25 to 40° C., and is preferably 30 to 38° C. and more preferably 33.5 to 37.5° C. The temperature at which the fragmented extracellular matrix undergoes solation from a gel state (sol transition temperature) may be, for example, 2 to 20° C., and is preferably 2 to 18° C. and more preferably 2.5 to 6.5° C. The gel transition temperature is a temperature at which as the temperature of the fragmented extracellular matrix is gradually increased from 4° C. to 40° C., the transmittance at 500 nm reaches an intermediate value of the maximum and minimum values.

The rate of gradual temperature increase may be 0.5° C./min or 1° C./min. The sol transition temperature is a temperature at which as the temperature of the fragmented extracellular matrix is gradually decreased from 40° C. to 4° C., the transmittance at 500 nm reaches an intermediate value of the maximum and minimum values. The rate of gradual temperature decrease may be 0.5° C./min or 1° C./min. The content of the fragmented extracellular matrix in the composition when the gel transition temperature and sol transition temperature are measured may be, for example, 2 to 5% by mass, 2% by mass, 3% by mass, or 5% by mass based on the total mass of the composition.

The fragmented extracellular matrix may be one that undergoes gelation from a sol state at 35.5° C.±2° C. and undergoes solation from a gel state at 4.5° C.±2° C.

Further, being in a sol state or in a gel state can be determined by measuring the transmittance at 500 nm. For example, determination can be made as being in a sol state if the transmittance at 500 nm is 40% or more, and as being in a gel state if the transmittance at 500 nm is less than 40%. The transmittance at 500 nm can be measured by using a method described later in Examples. The wavelength to measure the transmittance is not limited to 500 nm, and other wavelengths may be used. For example, the wavelength may be 550 nm, 600 nm, 650 nm, 700 nm, or 750 nm. The viscosity of a gel state is higher than that of a sol state.

A production method according to an embodiment of the invention makes it possible to produce an artificial adipose tissue, which includes a large-size three-dimensional tissue, with a thickness of 1 mm or more by using a relatively small number of cells.

The thickness of the artificial adipose tissue including the above three-dimensional tissue is preferably from 10 μm to 20 mm, more preferably from 100 μm to 15 mm, and particularly preferably from 1 mm to 10 mm. The lower limit of the thickness is not particularly limited and may be, for instance, 10 μm, 50 μm, 100 μm, 200 μm, 500 μm, 800 μm, 1 mm, 3 mm, or 5 mm. The upper limit of the thickness is not particularly limited and may be, for instance, 20 mm, 15 mm, 10 mm, 5 mm, 3 mm, 2 mm, 1.5 mm, or 1 mm. Such a three-dimensional tissue has a structure closer to the structure of a biological tissue, and is suitable as alternatives for experimental animals and graft materials.

As used herein, the “thickness of a three-dimensional tissue” means the distance between both ends in a direction vertical to the main surface when the three-dimensional tissue has a sheet shape or a rectangular parallelepiped shape. If the main surface has recess and/or a protrusion, the thickness means the distance across the thinnest portion of the main surface. When the three-dimensional tissue is spherical, the thickness means the diameter. Furthermore, when the three-dimensional tissue is elliptical, the thickness means the short diameter. If the three-dimensional tissue is substantially spherical or substantially elliptical with a recess and/or a protrusion on the surface, the thickness means the shortest distance between two points at which the surface intersects a line crossing the center of gravity of the three-dimensional tissue.

The artificial adipose tissue, which includes the three-dimensional tissue, has contraction rate during culturing of preferably 20% or less, more preferably 15% or less, and still more preferably 10% or less. The contraction rate may be calculated by, for instance, the following equation. In the equation, L1 denotes the length of the longest portion in the artificial adipose tissue including the three-dimensional tissue at day 1 of culturing; and L3 denotes the length of the corresponding portion in the artificial adipose tissue including the three-dimensional tissue at day 3 of culturing.

$$Contraction rate ( \% ) = [ ( L 1 - L 3 ) / L 1 ] \times 10 \; 0 .$$

In the above example, the contraction rate is calculated from the artificial adipose tissue at day 1 of culturing and the artificial adipose tissue at day 3 of culturing. However, the contraction rate may be calculated from the artificial adipose tissue at day 1 of culturing and the artificial adipose tissue at day 2 of culturing. Also, the contraction rate may be calculated from the artificial adipose tissue at day 1 of culturing and the artificial adipose tissue at day 5 of culturing. In addition, the contraction rate may be calculated from the artificial adipose tissue at day 1 of culturing and the artificial adipose tissue at day 8 of culturing.

The size of lipid droplet(s) may be used as an indicator for indicating the maturity of each adipocyte cultured. The lipid droplet is an intracellular organelle in which lipids such as triglyceride (neutral fat) and cholesterol are stored and has a liquid droplet-like shape such that the above lipids are covered by a phospholipid monolayer. In addition, an adipose tissue-specific protein (e.g., Perilipin) is expressed on the surface of the above phospholipid. Lipid droplets in mature adipocytes vary in size. For instance, if the average lipid droplet size is 20 μm or more, the adipocyte is substantially matured and can thus be a mature adipocyte.

The average size of lipid droplets in adipocytes in the artificial adipose tissue of this embodiment is preferably from 20 μm to 180 μm and more preferably from 100 μm to 180 μm. The above lipid droplet size may be 250 μm or less, 200 μm or less, 180 μm or less, or 150 μm or less. Also, the above lipid droplet size may be 15 μm or more, 30 μm or more, 50 μm or more, or 80 μm or more. In addition, the above lipid droplet size may be a number at day 7 of culturing, day 10 of culturing, day 14 of culturing, or day 21 of culturing.

As an indicator for indicating the maturity of adipocytes cultured, it is possible to use the expression profile of gene markers such as an adipogenesis marker and a lipolysis marker. Examples of the adipogenesis marker include Peroxisome Proliferator-Activated Receptor 12 (PPARγ2), Fatty Acid Binding Protein 4 (FABP4), and Glucose transporter type 4 (GLUT-4). PPARγ2 and FABP4 can be utilized as early adipogenesis markers, and GLUT-4 can be utilized as a late adipogenesis marker. Examples of the lipolysis marker include hormone-sensitive lipase (HSL) and Adipocyte Triglyceride Lipase (ATGL).

PPARγ2 is one of the most important transcription factors during differentiation of adipocytes. During adipogenesis, the level of expression of PPARγ2 reportedly increases and is then constant or decreases (B. Galateanu et al., Int. J. Mol. Sci. 2012 (13) 15881-15900 and A. Soukas et al., J. Biol. Chem. 2001 (276) 34167-34174). Accordingly, it is considered that adipogenesis is advanced more in a tissue at the PPARγ2 constant expression stage than, for instance, in a tissue at the PPARγ2 increasing stage. FABP4 is another early marker and is needed for membrane transport so as to traffic fatty acids. Accordingly, a tissue exhibiting a more typical linearly increasing FABP4 expression profile, for example, is considered to be at an earlier adipogenesis stage. Regarding the typical linearly increasing FABP4 expression profile, E. C. M. Mariman et al., Cell. Mol. Life Sci. 2010 (67) 1277-1292 can be consulted.

Insulin-stimulated GLUT-4 is a major glucose transporter in adipocytes. The level of expression of GLUT-4 is weak in a tissue at the initial stage of differentiation during adipogenesis (S.-W. Qian et al., BMC Dev. Biol. 2010 (10) 47). Thus, the adipogenesis differentiation in a tissue having a more rapid increase in the level of expression of GLUT4, for instance, is considered to be advanced more.

The artificial adipose tissue of this embodiment may contain cells other than an adipocyte and may contain an extracellular matrix-producing cell. The “extracellular matrix-producing cell” means a cell that secretes an extracellular matrix such as collagen. That is, this artificial adipose tissue may contain an endogenously produced extracellular matrix. Examples of the extracellular matrix-producing cell include mesenchymal cells such as fibroblasts, chondrocytes, or osteoblasts. Preferred are fibroblasts. Examples of the preferable fibroblasts include human dermal fibroblasts (NHDF), human cardiac fibroblasts (NHCF), and human gingival fibroblasts (HGF). Examples of the cells other than the adipocyte include vascular endothelial cells (e.g., human μmbilical vein endothelial cells (HUVECs)), cancer cells such as colon cancer cells (e.g., human colon cancer cells (HT29)) and liver cancer cells, cardiomyocytes (e.g., human iPS cell-derived cardiomyocytes (iPS-CMs)), epithelial cells (e.g., human gingival epithelial cells), lymphatic endothelial cells, neurons, hepatocytes, tissue stem cells, embryonic stem cells, induced pluripotent stem cells, adherent cells (e.g., immune cells), smooth muscle cells (e.g., aortic smooth muscle cells (Aorta-SMCs)), and keratinocytes (e.g., human epithelial keratinocytes).

The adipose tissue including the above three-dimensional tissue is applicable as alternatives for experimental animals, and graft materials etc. Specifically, the adipose tissue is applicable to, for instance, a cellulite, cosmetic assay screening for diabetes, etc., pharmaceutical screening for diabetes, etc., in vitro models for adipose tissue-related inflammatory disease or other pathologies, or tissue reconstruction after mastectomy or soft tissue lesion caused by trauma or tumorectomy.

### (Artificial Skin)

An artificial skin according to an embodiment of the invention includes multiple layers, one of which is a layer including the above-described artificial adipose tissue. Specifically, the artificial skin according to this embodiment includes a first layer and a second layer, wherein the above-described artificial adipose tissue is included as the first layer. This artificial skin may include at least the first layer and the second layer, further optionally include a third layer, a fourth layer, and a fifth layer, and may also further include a layer(s). The order of the respective layers may be determined arbitrarily. However, the layers are preferably stacked in the order of from the first layer to the second layer to the third layer. In this case, as long as the first layer, the second layer, and the third layer are stacked in this order, for instance, another layer may be included, without restriction, under the first layer, between the first layer and the second layer, between the second layer and the third layer, or over the third layer.

The respective layers in the artificial skin according to this embodiment are distinguishable based on the kind of cells included and the difference in the cell distribution, etc. For instance, the artificial skin may be stained with different colors depending on the kinds of cells. Then, each layer is indicated with a different color, so that each layer is distinguishable in cross-section pictures, etc. Also, for instance, all the cells included in the artificial skin are stained. Then, each layer is distinguishable because there is a difference in the density of cells stained in the cross-section pictures, etc.

The layers (e.g., the second, third, fourth, and fifth layers) other than the first layer contain cells. The cells may include the above-described adipocyte and may include a cell other than the adipocyte. The second layer preferably contains fibroblasts or keratinocytes, more preferably contains fibroblasts, and still more preferably contains human dermal fibroblasts or human cardiac fibroblasts. The third layer preferably contains fibroblasts or keratinocytes, more preferably contains keratinocytes, and still more preferably contains human epithelial keratinocytes. When the first layer is the bottom layer, it is preferable that the second layer contains fibroblasts and the third layer contains keratinocytes. When the layers are stacked in this way, an artificial skin closer to an in vivo human skin structure can be obtained.

In addition, the layers (e.g., the second, third, fourth, and fifth layers) other than the first layer may contain an extracellular matrix and may contain a fragmented extracellular matrix. The second layer preferably contains a fragmented extracellular matrix. Those described above may be used as the extracellular matrix and the fragmented extracellular matrix. Inclusion of the fragmented extracellular matrix allows for formation of a layer with suitable intercell distance.

The thickness of the artificial skin according to this embodiment is preferably from 10 μm to 20 mm, more preferably from 100 μm to 15 mm, still more preferably from 200 μm to 10 mm, and particularly preferably from 1 mm to 10 mm. The lower limit of the thickness is not particularly limited and may be, for instance, 10 μm, 50 μm, 100 μm, 200 μm, 500 μm, 800 μm, 1 mm, 3 mm, or 5 mm. The upper limit of the thickness is not particularly limited and may be, for instance, 20 mm, 15 mm, 10 mm, 5 mm, 3 mm, 2 mm, 1.5 mm, or 1 mm. Such an artificial skin has a structure closer to the structure of a biological tissue, and is suitable as alternatives for experimental animals and graft materials.

The “thickness of artificial skin” means the distance from the tissue surface of the top layer to the tissue bottom surface of the bottom layer. For instance, in the case of a three-layer tissue including epithelial, dermal, and subcutaneous tissues, the thickness means the distance from the surface of the epithelial tissue to the bottom surface of the bottom layer, namely, the subcutaneous tissue (the total thickness of the three-layer tissue).

The number of cells included in the artificial skin according to this embodiment may be 1×10⁶ or higher.

The first layer in the artificial skin of this embodiment includes the above-described artificial adipose tissue. The average size of lipid droplets in adipocytes in the artificial adipose tissue is preferably from 20 μm to 180 μm and more preferably from 100 μm to 180 μm. The above lipid droplet size may be 250 μm or less, 200 μm or less, 180 μm or less, or 150 μm or less. Also, the above lipid droplet size may be 15 μm or more, 30 μm or more, 50 μm or more, or 80 μm or more. In addition, the above lipid droplet size may be a number at day 7 of culturing, day 10 of culturing, day 14 of culturing, or day 21 of culturing.

The above artificial skin may be used as an alternative for in vivo skin and is applicable as an alternatives for experimental animals, graft materials, etc. Specifically, the artificial skin is applicable to, for instance, tests for evaluating skin permeability, skin safety, skin corrosion, skin irritancy, or the like of a compound, cosmetic assay screening, pharmaceutical screening, in vitro pathological models, and/or graft materials for plastic surgery or burn operation.

### (Method for Producing Artificial Adipose Tissue)

A method for producing an artificial adipose tissue according to an embodiment of the invention includes:
(1) contacting a population of cells containing an adipocyte with a fragmented extracellular matrix in an aqueous medium (hereinafter, sometimes referred to as step (1)); and
(2) culturing the cells contacted with the fragmented extracellular matrix (hereinafter, sometimes referred to as step (2)).

The “adipocyte” and the “fragmented extracellular matrix”, etc., are as described above.

The “a population of cells containing an adipocyte” in step (1) of this embodiment may contain cells other than an adipocyte and may contain an extracellular matrix-producing cell. The “extracellular matrix-producing cell” means a cell that secretes an extracellular matrix such as collagen. Examples of the extracellular matrix-producing cell include mesenchymal cells such as fibroblasts, chondrocytes, or osteoblasts. Preferred are fibroblasts. Examples of the preferable fibroblasts include human dermal fibroblasts (NHDF), human cardiac fibroblasts (NHCF), and human gingival fibroblasts (HGF). Examples of the cells other than the adipocyte include vascular endothelial cells (e.g., human μmbilical vein endothelial cells (HUVECs)), cancer cells such as colon cancer cells (e.g., human colon cancer cells (HT29)) and liver cancer cells, cardiomyocytes (e.g., human iPS cell-derived cardiomyocytes (iPS-CMs)), epithelial cells (e.g., human gingival epithelial cells), lymphatic endothelial cells, neurons, hepatocytes, tissue stem cells, embryonic stem cells, induced pluripotent stem cells, adherent cells (e.g., immune cells), smooth muscle cells (e.g., aortic smooth muscle cells (Aorta-SMCs)), and keratinocytes (e.g., human epithelial keratinocytes).

The “aqueous medium” means liquid using water as an essential component. As long as the fragmented collagen and the cells are present stably, the aqueous medium has no particular restriction. Examples include saline such as phosphate buffered saline (PBS) and liquid culture medium such as Dulbecco's Modified Eagle culture medium (DMEM), or vascular endothelial cell-specific culture medium (EGM2). A mixed culture medium, in which two different culture media are mixed, is acceptable as the liquid culture medium. From the viewpoint of decreasing a load against cells, it is preferable that the aqueous medium is liquid culture medium.

A procedure for contacting the population of cells containing an adipocyte with a fragmented extracellular matrix in an aqueous medium is not particularly limited. Examples include: a procedure for adding a fragmented extracellular matrix-containing dispersion to a culture liquid containing cells; a procedure for adding cells to a fragmented extracellular matrix-containing culture medium dispersion; or a procedure for separately adding a fragmented extracellular matrix and cells to a beforehand prepared aqueous medium.

The concentration of the fragmented extracellular matrix in the aqueous medium in step (1) may be determined, if appropriate, depending on the shape and the thickness of artificial adipose tissue including the three-dimensional tissue of interest, the size of cultureware, and so on. For instance, the concentration of the fragmented extracellular matrix in the aqueous medium in step (1) may be from 0.1 to 90 wt %, from 0.5 to 50 wt % and may be from 1 to 30 wt %.

The amount of the fragmented extracellular matrix in step (1) may be from 0.1 to 100 mg and may be from 1 to 50 mg with respect to 1×10⁵ cells.

The mass ratio between the fragmented extracellular matrix and the cells in step (1) is preferably from 1000:1 to 1:1, more preferably from 900:1 to 9:1, and still more preferably from 500:1 to 10:1.

When the adipocyte and the other cells are together used, the (cell count) ratio between the adipocyte and the other cells in step (1) may be from 99:1 to 9:1 and may be from 80:20 to 50:50.

The salt concentration of the aqueous medium in step (1) may be, for example, a salt concentration showing an ionic strength equivalent to 0.1 or more and 10 or less times that of phosphate-buffered saline (1×PBS). Specifically, the NaCl concentration may be 13.0 mM or more, 13.7 mM or more, 27.4 mM or more, 137 mM or more, 685 mM or more, 1370 mM or more. Additionally, the NaCl concentration may be 13.0 mM or more and 1400 mM or less, 13.7 mM or more and 1400 mM or less, 25.0 mM or more and 1400 mM or less, 27.4 mM or more and 1400 mM or less, 125.0 mM or more and 1400 mM or less, 137.0 mM or more and 1400 mM or less, or 625.0 mM or more and 1400 mM or less. Furthermore, the NaCl concentration may be 13.0 mM or more and 1370 mM or less, 13.7 mM or more and 1370 mM or less, 25.0 mM or more and 1370 mM or less, 27.4 mM or more and 1370 mM or less, 125.0 mM or more and 1370 mM or less, 137.0 mM or more and 1370 mM or less, or 625.0 mM or more and 1370 mM or less. The salt concentration of the aqueous medium in step (1) is preferably one that can provide sufficient ionic strength to induce the thermally reversible sol-gel transition of the fragmented extracellular matrix, which can be achieved within the aforementioned range.

The pH of the aqueous medium in step (1) may be, for example, neutral. Specifically, it may be, for example, in the range of pH 6.0 or more and 8.0 or less, and preferably in the range of pH 7.2 or more and 7.4 or less.

The production method may further include, between step (1) and step (2), the step of subjecting the population of the cells containing the adipocyte and the fragmented extracellular matrix in the aqueous medium to precipitate. Such a step may be conducted to produce a more uniform distribution of the cells and the fragmented extracellular matrix in the artificial adipose tissue including the three-dimensional tissue. The specific procedure is not particularly limited. Examples include a procedure in which a culture liquid containing the fragmented extracellular matrix and the cells is centrifuged.

In step (1), the step may be implemented by forming a cell layer in the aqueous medium and then by contacting it with the fragmented extracellular matrix. The cell layer may be formed before the contact with the fragmented extracellular matrix to produce an artificial adipose tissue including a three-dimensional tissue, a lower layer of which has a high cell density. For instance, the cell layer containing an extracellular matrix-producing cell is formed before the contact with the fragmented extracellular matrix. This makes it possible to produce an artificial adipose tissue including a three-dimensional tissue having a high cell density in the lower layer portion including the population of the cells containing an extracellular matrix-producing cell. Depending on the kinds of cells used, this method allows for production of an artificial adipose tissue closer to in vivo one.

In this embodiment, the method for producing a three-dimensional tissue optionally includes, as step (3) after step (2), the step of further contacting it with cells and culturing the cells. These cells may be the same as or different from the cells used in step (1). For instance, when the cells used in step (1) contain cells other than an extracellular matrix-producing cell, the cells used in step (3) may contain an extracellular matrix-producing cell. Also, for instance, when the cells used in step (1) contain an extracellular matrix-producing cell, the cells used in step (3) may contain cells other than an extracellular matrix-producing cell. Both the cells used in step (1) and the cells used in step (3) may contain an extracellular matrix-producing cell. Both the cells used in step (1) and the cells used in step (3) may contain cells other than an extracellular matrix-producing cell. The above step (3) allows for production of an artificial adipose tissue including a three-dimensional tissue with a double layer structure. Substantially the same method may be used to produce an artificial skin including multiple layers. A method for producing this artificial skin will be described below.

In step (2), a procedure for culturing the cells contacted with the fragmented extracellular matrix is not particularly limited and may be implemented using a suitable culture protocol depending on the kind of cells cultured. For instance, the culturing temperature may be from 20° C. to 40° C. and may be from 30° C. to 37° C. The pH of the culture medium may be from 6 to 8 and may be from 7.2 to 7.4. The production method according to this embodiment does not require a culture medium with a complicated composition as used for conventional three-dimensional culture or three-dimensional tissue production. A culture medium, such as DMEM, that can be prepared easily may be used. The culture medium is not particularly limited, and a suitable culture medium can be selected depending on the kinds of cells cultured. Examples of the culture medium include Eagle's MEM culture medium, DMEM, Modified Eagle Medium (MEM), Minimum Essential culture medium, RPMI, or GlutaMax culture medium. The culture medium may be serum-containing medium or serum-free medium. A mixed culture medium, in which two different culture media are mixed, is acceptable as the culture medium. In addition, the production method according to this embodiment makes it possible to produce a fully differentiated artificial adipose tissue in a much shorter culturing period than that of conventional two-dimensional culture. Thus, the culturing period in step (2) may be, for instance, from 10 days to 60 days, from 10 days to 30 days, from 13 days to 25 days, or from 14 days to 17 days. Provided that the above culturing period just represents a period required for obtaining a fully differentiated adipose tissue and is not set to prohibit culturing of the period or longer.

The cell density in the culture medium in step (2) may be determined, if appropriate, depending on the shape and the thickness of three-dimensional tissue of interest, the size of cultureware, and so on. For instance, the cell density in the culture medium in step (2) may be from 1 to 10⁸ cells/mL and may be from 103 to 10⁷ cells/mL. In addition, the cell density in the culture medium in step (2) may be the same as that in the aqueous medium in step (1).

The artificial adipose tissue, which includes the three-dimensional tissue produced by the production method according to this embodiment, has the contraction rate during culturing of preferably 20% or less, more preferably 15% or less, and still more preferably 10% or less. The contraction rate may be calculated by, for instance, the following equation. In the equation, L1 denotes the length of the longest portion in the three-dimensional tissue at day 1 of culturing; and L3 denotes the length of the corresponding portion in the three-dimensional tissue at day 3 of culturing.

$$Contraction rate ( \% ) = [ ( L 1 - L 3 ) / L 1 ] \times 10 \; 0 .$$

In the above example, the contraction rate is calculated from the artificial adipose tissue at day 1 of culturing and the artificial adipose tissue at day 3 of culturing. The contraction rate may be calculated from the artificial adipose tissues at any culturing time points including the end point of culturing. For instance, the contraction rate may be calculated from the artificial adipose tissue at day 1 of culturing and the artificial adipose tissue at day 2 of culturing. Also, the contraction rate may be calculated from the artificial adipose tissue at day 1 of culturing and the artificial adipose tissue at day 5 of culturing. In addition, the contraction rate may be calculated from the artificial adipose tissue at day 1 of culturing and the artificial adipose tissue at day 8 of culturing.

The method for producing an artificial adipose tissue may include (4) a step of cooling the artificial adipose tissue formed to allow the fragmented extracellular matrix to undergo solation (step (4)), and (5) a step of removing after step (4) the fragmented extracellular matrix after undergoing solation. Cooling of the artificial adipose tissue may be performed in an aqueous medium.

Through inclusion of step (4) and step (5) in the method for producing an artificial adipose tissue, at least a part of extracellular matrix derived from the fragmented extracellular matrix (exogenous extracellular matrix) can be removed after formation of the artificial adipose tissue.

In step (4), the artificial adipose tissue is cooled to allow the fragmented extracellular matrix to undergo solation. Cooling of the artificial adipose tissue may be performed in an aqueous medium. Cooling of the artificial adipose tissue may be performed by decreasing the temperature of a culture solution containing the artificial adipose tissue and an aqueous medium. The temperature in cooling the artificial adipose tissue (cooling temperature) may be appropriately set according to the type of the aqueous medium and so forth. The cooling temperature may be, for example, 15° C. or less, or 4° C., or over 0° C.

In step (5), the fragmented extracellular matrix after undergoing solation (in a sol state) is removed. Thereby, at least a part or all of the fragmented extracellular matrix, which is an exogenous extracellular matrix component, can be removed from the artificial adipose tissue.

Removal of the fragmented extracellular matrix in a sol state can be performed, for example, by suspending the artificial adipose tissue in which a part or all of the fragmented extracellular matrix is in a sol state in an aqueous medium (e.g., phosphate-buffered saline), and centrifuging the suspension to remove the supernatant.

After culturing the artificial adipose tissue, which is formed with an extracellular matrix that exhibits thermally reversible sol-gel transition, for a certain period of time under normal physiological conditions (pH 7 to 8, 37° C.), gelation has proceeded to some degree with cells included in the aqueous medium. By cooling the artificial adipose tissue in an aqueous medium, the fragmented extracellular matrix, which is an exogenous extracellular matrix component, can be allowed to undergo solation and removed. Through removal of the exogenous extracellular matrix among the constituent components of the artificial adipose tissue, a more in-vivo-like tissue without any exogenous component can be obtained.

The residual percentage of the artificial adipose tissue, which includes the three-dimensional tissue produced by the production method according to this embodiment, after trypsin treatment at a trypsin concentration of 0.25%, a temperature of 37° C., a pH of 7.4, and a reaction time of 15 min is preferably 70% or higher, more preferably 80% or higher, and still more preferably 90% or higher. Such a three-dimensional tissue is not susceptible to enzymatic degradation during or after culturing and is thus stable. The above residual percentage may be calculated, for instance, from the masses of the three-dimensional tissue before and after the trypsin treatment.

The residual percentage of the artificial adipose tissue, which includes the three-dimensional tissue produced by the production method according to this embodiment, after collagenase treatment at a collagenase concentration of 0.25%, a temperature of 37° C., a pH of 7.4, and a reaction time of 15 min is preferably 70% or higher, more preferably 80% or higher, and still more preferably 90% or higher. Such a three-dimensional tissue is not susceptible to enzymatic degradation during or after culturing and is thus stable.

In addition, the production method according to this embodiment allows for production of an artificial adipose tissue including a stable three-dimensional tissue in which cells are evenly distributed. Further, the artificial adipose tissue produced by the production method according to this embodiment can be maintained for a longer period than that produced by conventional two-dimensional culture. Furthermore, the production method according to this embodiment allows for production of an artificial adipose tissue including a large-size three-dimensional tissue having a relatively small number of cells and a thickness of 1 mm or more.

### (Method for Producing Artificial Skin)

A method for producing an artificial skin according to an embodiment of the invention includes:
forming a first layer, including (1) contacting first population of cells containing an adipocyte with a first fragmented extracellular matrix in an aqueous medium (hereinafter, referred to as step (1)′), and
(2) culturing the first population of cells contacted with the first fragmented extracellular matrix, thereby (hereinafter, sometimes referred to as step (2)′); and
forming a second layer, including (3) further contacting the first layer with second population of cells and culturing the second cells (hereinafter, sometimes referred to as step (3)′).

Substantially the same procedures as describe above (for the artificial adipose tissue production method) are implemented in steps (1)′ and (2)′. The aqueous medium, the first population of cells containing an adipocyte, the second population of cells, and the first fragmented extracellular matrix may be represented likewise by the above-described aqueous medium, the population of cells containing an adipocyte, and the fragmented extracellular matrix.

The procedure for forming the first layer in step (2)′ is not particularly limited. As long as the thickness of cells cultured is a desired thickness, the layer may be considered to be formed. Step (3)′ is not necessarily started after completion of the first layer formation in step (2)′. It is possible to start step (3)′ during the culturing in step (2)′.

The average length of the first fragmented extracellular matrix is preferably from 100 nm to 200 μm, more preferably from 22 μm to 200 μm, and still more preferably from 100 μm to 200 μm. The average diameter of the fragmented extracellular matrix is preferably from 50 nm to 30 μm, more preferably from 4 μm to 30 μm, and still more preferably from 20 μm to 30 μm.

Examples of the first fragmented extracellular matrix include substantially the same one as mentioned above (for the artificial adipose tissue). It is preferable that the first fragmented extracellular matrix contains fragmented collagen. It is more preferable that the first fragmented extracellular matrix is fragmented collagen.

In addition, the production method according to this embodiment makes it possible to produce a fully differentiated adipose tissue (the first layer) in a much shorter culturing period than that of conventional two-dimensional culture. Thus, the culturing period in step (2)′ may be, for instance, from 10 days to 60 days, from 10 days to 30 days, from 13 days to 25 days, or from 14 days to 17 days. Provided that the above culturing period just represents a period required for obtaining a fully differentiated adipose tissue and is not set to prohibit culturing of the period or longer.

The second population of cells may be the same as or different from the first population of cells. Substantially the same procedures as describe above (for the artificial adipose tissue production method) are implemented in the cell contact procedure and the culturing protocol.

Examples of the cells used as the second population of cells include substantially the same cells as mentioned above (for the artificial adipose tissue). Here, it is preferable that the second population of cells contain fibroblasts. When the second layer is a layer containing differentiated fibroblasts, the structure can be closer to that of in vivo skin.

In addition, step (3)′ is preferably a step comprising contacting the second population of cells with a second fragmented extracellular matrix on the first layer in an aqueous medium and culturing the population of second cells. Examples of the second extracellular matrix include substantially the same one as mentioned above (for the artificial adipose tissue). Inclusion of the fragmented extracellular matrix allows for formation of a layer with suitable intercell distance. It is preferable that the second fragmented extracellular matrix contains fragmented collagen. It is more preferable that the second fragmented extracellular matrix is fragmented collagen.

The artificial skin production method according to this embodiment may further comprise forming a third layer including further contacting the second layer with third population of cells and culturing the third population of cells.

Examples of the cells used as the third population of cells include substantially the same cells as mentioned above (for the artificial adipose tissue). It is preferable that the third cells contain keratinocytes. When the third layer is a layer containing differentiated keratinocytes, the structure can be closer to that of in vivo skin.

The method for producing an artificial skin may include (4)′ a step of cooling the artificial skin formed to allow the fragmented extracellular matrix to undergo solation (step (4)′), and (5)′ a step of removing after step (4)′ the fragmented extracellular matrix after undergoing solation (step (5)′). Cooling of the artificial skin may be performed in an aqueous medium. The specific embodiments of steps (4)′ and (5)′ are the same as those of steps (4) and (5) described above.

As likewise described above (for the artificial skin), the artificial skin production method according to this embodiment makes it possible to produce an artificial skin with a thickness close to that of in vivo skin in a much shorter culturing period than that of conventional two-dimensional culture.

In addition, the artificial skin production method according to this embodiment allows for production of an artificial skin including, as the first layer, an artificial adipose tissue including a stable three-dimensional tissue in which cells are evenly distributed. Further, in the artificial adipose tissue in the artificial skin produced by the production method according to this embodiment, mature adipocytes can be maintained for a longer period than those during conventional two-dimensional culture. Furthermore, the production method according to this embodiment makes it possible to produce an artificial skin having a relatively small number of cells and a thickness of 1 mm or longer, which thickness is close to that of in vivo skin.

### <Agent for Adipocyte Culture>

An agent for adipocyte culture according to an embodiment of the invention contains a fragmented extracellular matrix. In the agent for adipocyte culture according to this embodiment, the average length of the fragmented extracellular matrix may be from 100 nm to 200 μm. The average diameter of the fragmented extracellular matrix may be from 50 nm to 30 μm. Here, the length of 95% of the fragmented extracellular matrix with respect to the whole extracellular matrix may be ranged from 100 nm to 200 μm. Here, the diameter of 95% of the fragmented extracellular matrix with respect to the whole extracellular matrix may be ranged from 50 nm to 30 μm.

The “agent for adipocyte culture” means a reagent for culturing adipocytes. The agent for adipocyte culture may be in a powder state or may be in a dispersion state in which the fragmented extracellular matrix is dispersed in an aqueous medium. Examples of the kind, size, and production method of the fragmented extracellular matrix and usage of the above culture agent include substantially the same ones as designated above (for the artificial adipose tissue) and (for the artificial adipose tissue production method). Inclusion of the fragmented extracellular matrix can promote differentiation of adipocytes. Thus, the agent for adipocyte culture may be understood as a differentiation promoter. In addition, inclusion of the fragmented extracellular matrix is unlikely to cause mature adipocytes to be dedifferentiated. Thus, the agent for adipocyte culture may also be understood as a mature adipocyte dedifferentiation inhibitor (agent for maintaining mature adipocytes).

Further, the culture agent according to this embodiment makes it possible to form a tissue having less tissue contraction due to long-term culturing even when a tissue having a thickness of, for instance, 1 mm or more is formed. The culture agent can exert a differentiation-promoting effect and a dedifferentiation inhibitory effect to form a tissue, the shape of which is stable for a long period of time.

### (Method for Evaluating Skin Permeability of Compound)

A method for evaluating skin permeability of a compound according to an embodiment of the invention comprises:
providing an artificial skin;
contacting the artificial skin with a test compound;
measuring skin permeability of the test compound using the artificial skin contacted with the test compound; and
comparing between the skin permeability of the test compound and a reference value.

As the artificial skin, it is possible to use an artificial skin according to the embodiment as described above (for the artificial skin) or an artificial skin produced by the production method according to the embodiment as described above (for the artificial skin production method). As stated above, this artificial skin has a structure and a thickness similar to those of in vivo skin and may be used, as an in vivo skin alternative, for a skin permeability test of a compound.

The test compound is not particularly limited and any test compound can be used as long as the compound is used in a regular skin permeability test.

A contact procedure for contacting the artificial skin with the test compound is not particularly limited. In the case of the test compound being liquid, the test compound may be, for example, directly applied or sprayed on the surface of the artificial skin. In the case of the test compound being solid, a solution in which the test compound has been dissolved in a solvent may be used to likewise apply or spray on the surface of the artificial skin.

The skin permeability of the test compound may be measured based on a skin permeability test protocol well-known to those skilled in the art. For instance, safety of a cosmetic/quasi drug of interest may be evaluated and tested in accordance with the guidance in Notification No. 1115-1 of the Central Pharmaceutical Affairs Council (Nov. 15, 2016).

The reference value may be set, if appropriate, based on the kind and the concentration of the test compound. For instance, when the reference value is one point and the skin permeability is higher than the reference value, the skin permeability can be determined as high. When the skin permeability is lower than the reference value, the skin permeability can be determined as low. In addition, the reference value may be set as, for instance, a range, within which the skin permeability is ranked.

Hereinbelow, the invention will be specifically described in detail with reference to Examples. However, the Examples do not restrict the scope of the invention.

### EXAMPLES

(Example 1: Production of Artificial Adipose Tissue Using Mouse Mature Adipocytes)

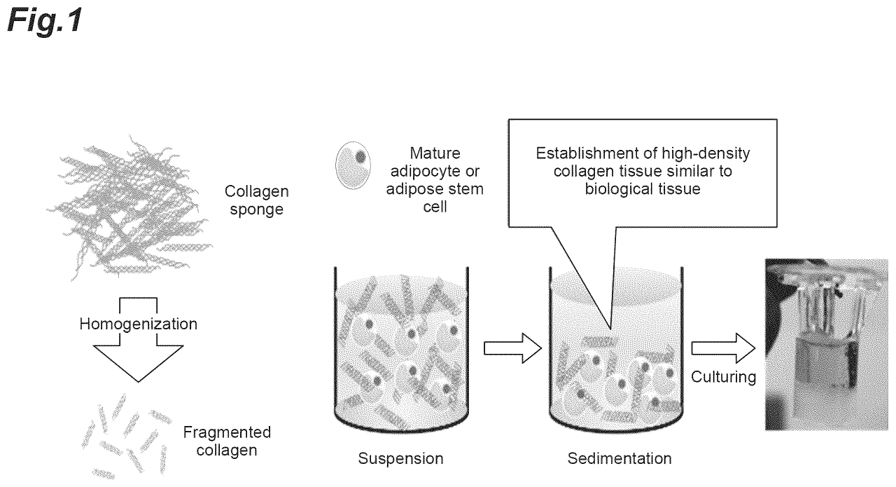

The present inventors developed a method for producing a three-dimensional tissue using collagen microfiber so as to increase collagen density in the tissue (FIG. 1). An artificial adipose tissue using mature adipocytes was produced as illustrated below in the schematic diagram of FIG. 1.

Lyophilized, pig skin-derived type I collagen manufactured by NH Foods Ltd. was dispersed in 10× conc. phosphate buffered saline (×10 PBS) and homogenized using a homogenizer for 5 min to yield fragmented collagen with a diameter of about 4.4 μm and a length of about 22.5 μm. The resulting fragmented collagen was washed with serum-free culture medium (DMEM) to prepare a fragmented collagen-containing culture medium dispersion.

The fragmented collagen was dispersed at a concentration of 10 mg/ml in serum-containing culture medium (DMEM). Then, 1 mL of the resulting dispersion (corresponding to about 10 mg fragmented collagen) and 1×10⁶ primary mature mouse adipocytes in DMEM were mixed in a 24-well plate transwell (manufactured by IWAKI, Inc.). Twenty-four hours after the seeding, the transwell was placed in a 6-well plate (manufactured by IWAKI, Inc.) containing 6 mL of culture medium (DMEM culture medium, manufactured by NACALAI TESQUE, INC.). The mature adipocytes were cultured until day 14 while the culture medium was changed every 4 days. After the start of culturing, the mixture containing the cells and the fragmented collagen was just subject to precipitate. Because of no centrifugation, the thickness decreased, over the culturing process, in the gravity direction and became stable to produce a three-dimensional tissue with a thickness of about 2 mm after 14 days of culturing. An artificial adipose tissue including the resulting three-dimensional tissue was subjected to hematoxylin/eosin (HE) staining and/or immunostaining with an anti-perilipin antibody. Then, histological evaluation was conducted. In addition, Nile red was used to stain the lipids. The viability was assessed using a Live/Dead kit (manufactured by Thermo Fisher Scientific, Inc.). The fat vesicles were analyzed and the Live/Dead was measured by ImageJ software (provided by National Institutes of Health in the U.S.). The fat vesicle diameters of 100 fat vesicles were measured by electron microscopy at day 7 of culturing and day 14 of culturing, and were compared to those of adipocytes in an artificial adipose tissue produced by conventional two-dimensional culture.

The method for producing an artificial adipose tissue by conventional two-dimensional culture was carried out as follows. First, 3×10⁵ (cells/well) mouse primary mature adipocytes were seeded on a 6-well plate (manufactured by IWAKI, Inc.) and then cultured. The culturing was continued in 2 mL of culture medium (DMEM culture medium, manufactured by NACALAI TESQUE, INC.). The culture medium was changed every 3 days. Note that mature adipocytes do not adhere to the bottom surface of dish because lipid droplets cause floatation effects. Thus, a commercially available cover glass was floated on the culture medium, and the cells was adhered underneath during the culturing.

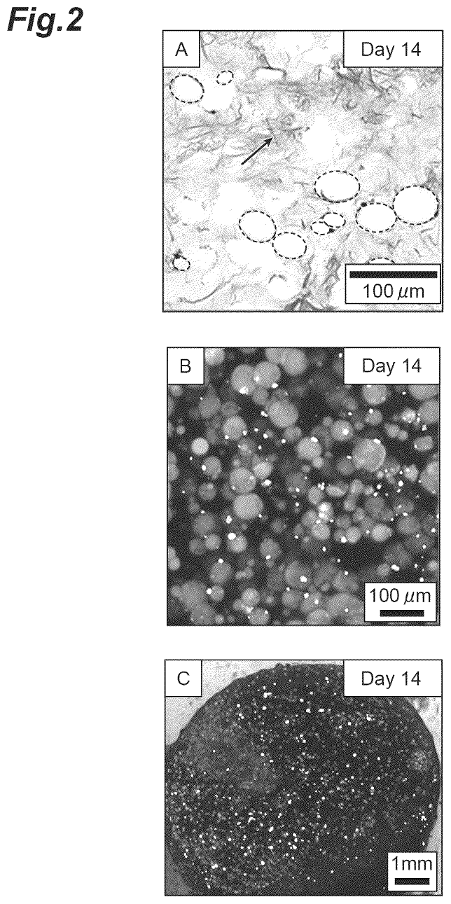

The HE staining revealed the presence of high-density fragmented collagen in the three-dimensional tissue surrounding the mature adipocytes (the arrow in FIG. 2A). The mature adipocytes each had a small cytoplasm and a nucleus, and a typical round uniocular shape (FIG. 2A).

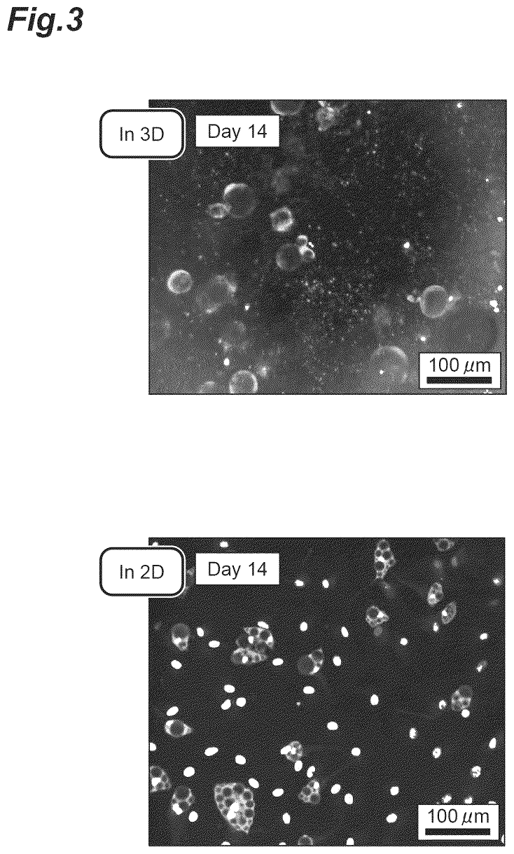

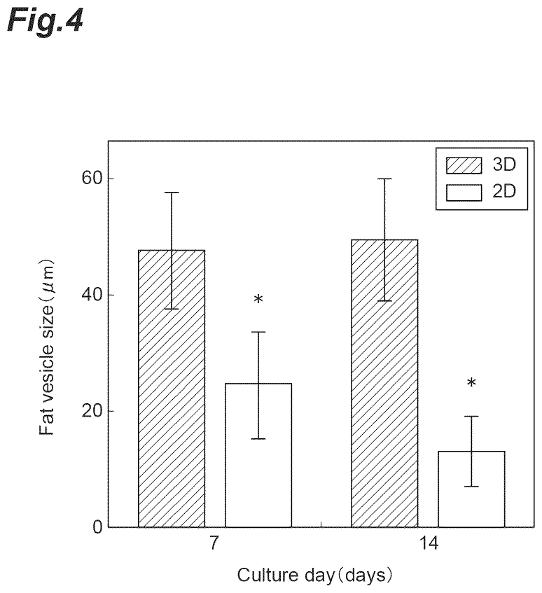

The Nile red lipid staining demonstrated a lipid droplet(s) in the cytoplasm. A uniocular lipid droplet, which indicates a mature adipocyte, was found, indicating a homogenous, high redifferentiation of mature adipocytes in the three-dimensional tissue (FIG. 2B). Even in the case of long-term culturing of 14 days or longer, the mature adipocytes in the artificial adipose tissue were well maintained at high viability of from 98.5% at day 2 to 94.6% at day 14 (in FIG. 2C, gray indicates living cells and white indicates dead cells). The results of immunostaining with an anti-perilipin antibody demonstrated that the mature adipocytes in the three-dimensional tissue at day 14 of culturing maintained their larger uniocular shape (a fat vesicle with a size of 54±8 μm at day 0) than adipocytes in an artificial adipose tissue produced by conventional two-dimensional culture (FIG. 3). By contrast, the mature adipocytes in the artificial adipose tissue produced by conventional two-dimensional culture were dedifferentiated to a fibroblast-like cell morphology and had vesicles 3.7 times significantly smaller than those of the mature adipocytes in the three-dimensional tissue at day 14 of culturing (in FIG. 4, n>100 fat vesicles were counted; t-test). Also, in the artificial adipose tissue including the three-dimensional tissue containing fragmented collagen, the size of fat vesicles was maintained from day 7 to day 14. However, in the artificial adipose tissue produced by conventional two-dimensional culture, the size of fat vesicles decreased from day 7 to day 14. This indicates that the time to dedifferentiation was longer in the three-dimensional tissue containing fragmented collagen than in the artificial adipose tissue produced by conventional two-dimensional culture, so that the long-term maintenance was possible and the culturing period of the mature adipocytes was successfully extended by at least 1 week or longer.

### Example 2: Production of Artificial Adipose Tissue Using Human Adipose Stem Cells

An artificial adipose tissue using adipose stem cells was produced as illustrated below in the schematic diagram of FIG. 1.

Lyophilized, pig skin-derived type I collagen manufactured by NH Foods Ltd. was dispersed in 10× conc. phosphate buffered saline (×10 PBS) and homogenized using a homogenizer for 5 min to yield fragmented collagen with a diameter of about 4.4 μm and a length of about 22.5 μm. The resulting fragmented collagen was washed with serum-free medium (DMEM) to prepare a fragmented collagen-containing culture medium dispersion.

The fragmented collagen was dispersed at a concentration of 10 mg/ml in serum-containing culture medium (DMEM). Then, 300 μL of the resulting dispersion (corresponding to about 3 mg fragmented collagen) and 5×10⁵ human adipose stem cells in DMEM were mixed in a 96-well plate insert (manufactured by ACEA Bioscience, Inc.). Twenty-four hours after the seeding, 200 μL of culture medium (DMEM culture medium, manufactured by NACALAI TESQUE, INC.) was added. The culture medium was changed every 2 days and the culturing was conducted for 19 days. Of 19 days, 2 days correspond to a growth period and the subsequent 17 days correspond to a differentiation period. After the start of culturing, the mixture containing the cells and the fragmented collagen was just subject to precipitate. Because of no centrifugation, the thickness decreased, over the culturing process, in the gravity direction and became stable to produce a three-dimensional tissue with a thickness of about 2 mm after 14 days of culturing. An artificial adipose tissue including the three-dimensional tissue at day 17 of differentiation (day 19 of culturing) was subjected to hematoxylin/eosin (HE) staining and/or immunostaining with an anti-perilipin antibody. Then, histological evaluation was conducted.

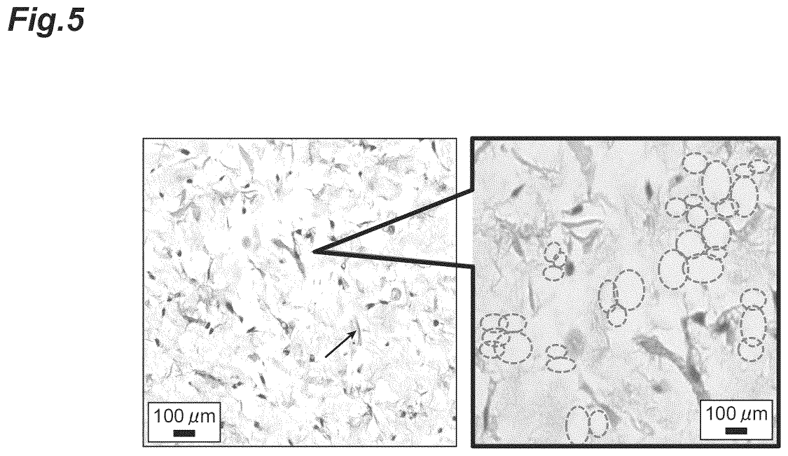

The HE staining revealed the presence of high-density fragmented collagen in the three-dimensional tissue (the arrow in FIG. 5). A large round uniocular lipid droplet, which indicates a mature adipocyte, was found (a magnified view in FIG. 5). Among them, large mature adipocytes with a size of more than 100 μm were also found.

A method for producing an artificial adipose tissue by conventional two-dimensional culture was carried out as follows. First, 1×10⁴ (cells/well) human adipose stem cells were seeded on a 24-well plate (manufactured by IWAKI, Inc.) and then cultured. During 2 days after the seeding, 500 μL of D-MEM was used for the culturing. After their growth, the culture medium was changed to a differentiation-promoting culture medium. The differentiation-promoting culture medium was prepared by adding PGM-2 Singlequot Kit PT-9502 (manufactured by LONZA) to D-MEM and contained growth aids (Indomethacin, 3-isobutyl-1-methyl xanthine, dexamethasone, and insulin). The culturing was conducted in 500 μL, and the culture medium was changed once a week.

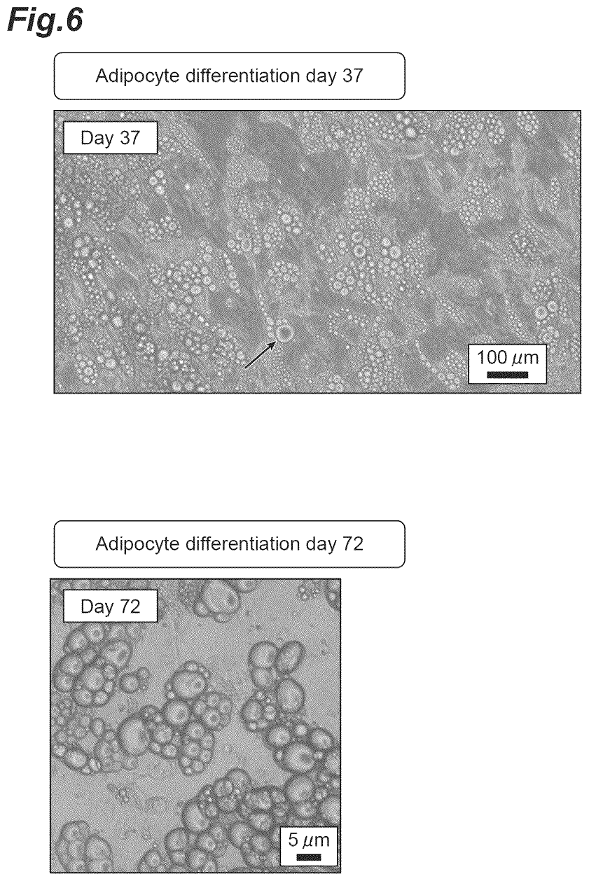

In the artificial adipose tissue produced by conventional two-dimensional culture, fat vesicles in a liquid droplet state were found here and there even at a time point of day 37 of differentiation (FIG. 6). In addition, when examined by Nile red lipid staining, only small lipid droplets were found. Mature adipocytes with a size of more than 100 μm, like in the three-dimensional tissue containing fragmented collagen, were not found.

Further, in the artificial adipose tissue produced by conventional two-dimensional culture, fat vesicles in a liquid droplet state were found here and there even at a time point of day 72 of differentiation; and any of the adipocytes were not differentiated (FIG. 6). Furthermore, as the fat content increased, the adipocytes detached and floated more in the culture liquid. As a result, it was difficult to change the culture liquid.

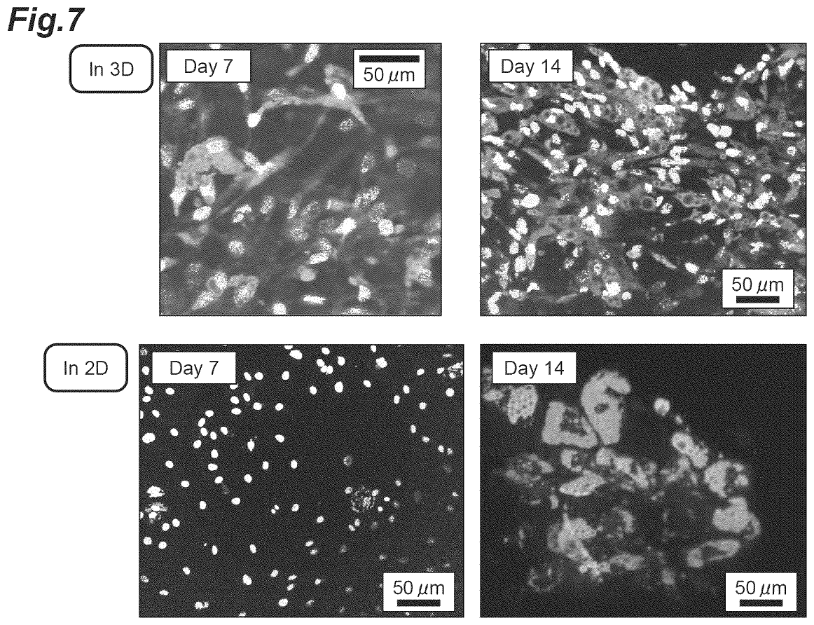

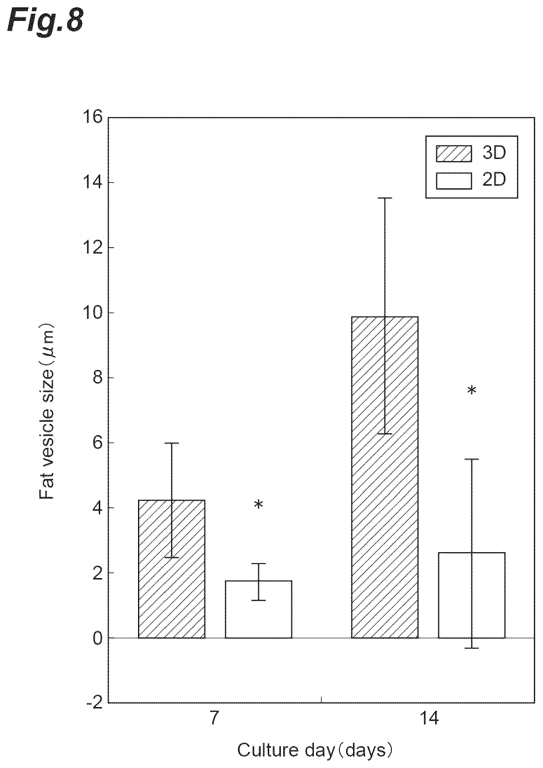

The results of immunostaining with an anti-perilipin antibody demonstrated that a larger uniocular shape (fat vesicle with a size of 4 μm±2 μm at day 7) was formed in the adipose stem cells in the three-dimensional tissue at day 7 of culturing than in adipose stem cells in an artificial adipose tissue produced by conventional two-dimensional culture (FIG. 7). Then, the artificial adipose tissue produced by conventional two-dimensional culture had vesicles 2 times significantly smaller than those of the adipocytes in the three-dimensional tissue at day 7 of culturing (in FIG. 8, n>100 fat vesicles were counted; t-test). Here, in the artificial adipose tissue including the three-dimensional tissue containing fragmented collagen, the size of fat vesicles was doubled or more (10 μm±4 μm) from day 7 to day 14. However, in the artificial adipose tissue produced by conventional two-dimensional culture, the size of fat vesicles had almost no change from day 7 to day 14.

The above has demonstrated that differentiation of the artificial adipose tissue including the three-dimensional tissue containing fragmented collagen progresses more rapidly than that of the artificial adipose tissue produced by conventional two-dimensional culture.

### Example 3: Production of Artificial Skin Using Mature Adipocytes

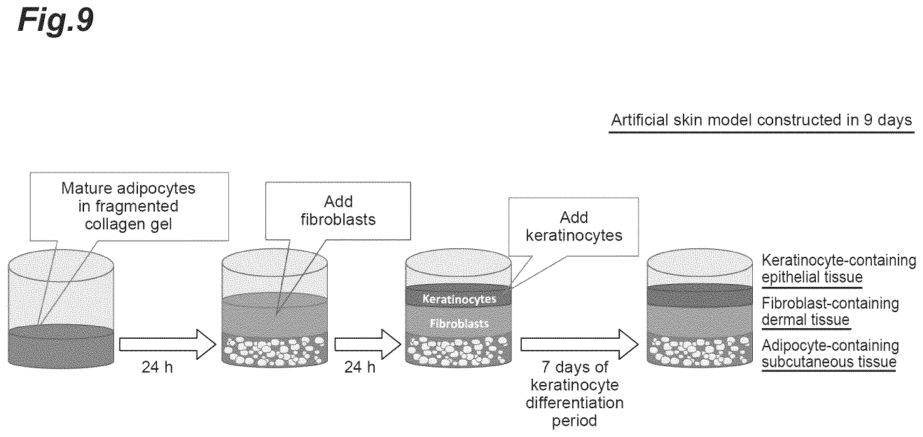

An artificial skin using mature adipocytes was produced as illustrated below in the schematic diagram of FIG. 9.

Lyophilized, pig skin-derived type I collagen manufactured by NH Foods Ltd. was dispersed in 10× conc. phosphate buffered saline (×10 PBS) and homogenized using a homogenizer for 5 min to yield fragmented collagen with a diameter of about 4.4 μm and a length of about 22.5 μm. The resulting fragmented collagen was washed with serum-free medium (DMEM) to prepare a fragmented collagen-containing culture medium dispersion.

The fragmented collagen was dispersed at a concentration of 10 mg/ml in serum-containing culture medium (DMEM). Then, 1 mL of the resulting dispersion (corresponding to about 10 mg fragmented collagen) and 1×10⁶ rat mature adipocytes, which had been collected from a rat subcutaneous tissue, in DMEM were mixed in a 24-well plate transwell (manufactured by IWAKI, Inc.). This plate was placed in an incubator, and the culturing was conducted for 24 h.

Next, a 24-well insert was coated with a 0.04 mg/mL fibronectin solution (#F2006-5G, manufactured by Sigma, Inc.) in PBS (0.04 μL/insert), and was incubated at 37° C. for 20 min. Then, 1 mL of the above fragmented collagen-containing culture medium dispersion (corresponding to about 10 mg of fragmented collagen) and 5×10⁵ human dermal fibroblasts (NHDF) were mixed, and 100 μL of the mixture was aliquoted in the 24-well insert. The plate was placed in an incubator, and the culturing was conducted for 24 h.

Next, the culture medium over the NHDF-containing collagen gel in the insert was aspirated. Then, the gel was coated with 0.04 mg/mL collagen IV solution in PBS (0.04 μL/insert) and was incubated at 37° C. for at least 20 min. The collagen IV solution added onto the NHDF-containing collagen gel was aspirated. After that, 2×10⁶ normal human epithelial keratinocytes (#KK-4009, 1 vial=500000 cells, manufactured by KURABO, Inc.) were added onto the NHDF-containing collagen gel. Subsequently, 1 mL of DMEM 5% FBS: EpiLife (#C-2517A, manufactured by Invitrogen, Inc.) (1:1) culture medium was added to the outside of the insert. After 1 h, another 1 mL was added to the outside of the insert.

The culture media on the inside and the outside were gently aspirated. Next, ascorbic acid was diluted 100-fold in DMEM 5% FBS: EpiLife (1:1) culture medium, which was then added, as 500 μL of differentiation medium, to the outside of the insert. No culture medium was added to the inside of the insert. The culture medium outside the insert was changed every day until differentiation day 7.

The above method required just 9 days from the start of adipose tissue culturing (24 h of adipose tissue culturing+24 h of fibroblast culturing+7 days of keratinocyte differentiation) so as to be able to produce an artificial skin with a three-layer structure including a bottom adipose tissue layer, an intermediate fibroblast layer, and a top keratinocyte layer (FIG. 9).

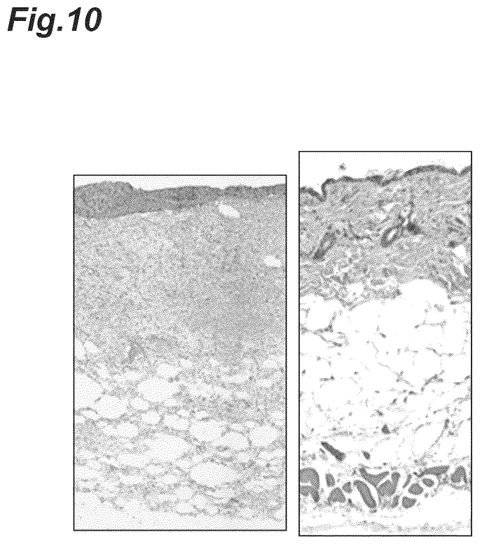

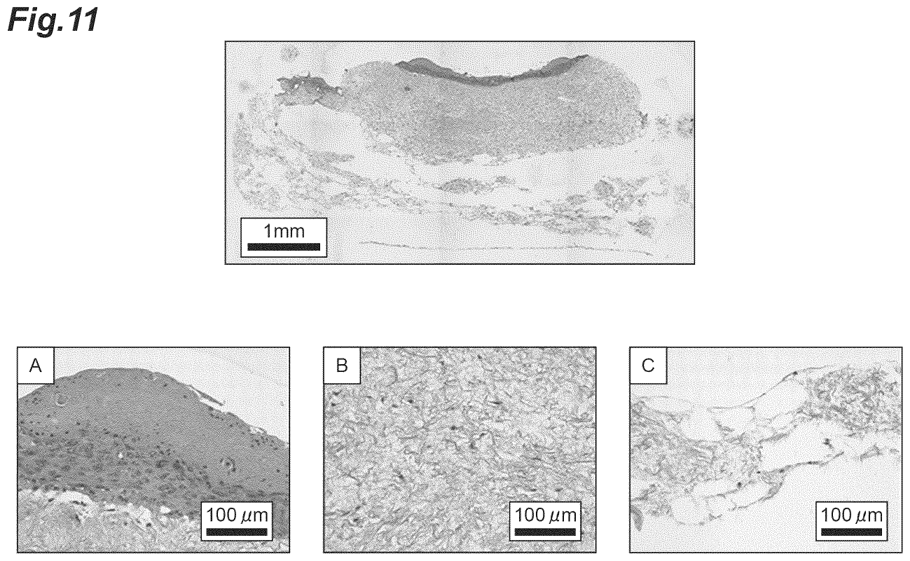

The artificial skin as so produced at keratinocyte differentiation day 7 was subjected to hematoxylin/eosin (HE) staining for histological evaluation. The above artificial skin had an appropriate intercell distance in any of the layers including the bottom adipocyte-containing layer (subcutaneous tissue), the intermediate fibroblast-containing layer (dermal tissue), and the top keratinocyte-containing layer (epithelial tissue), so that the artificial skin had a structure close to that of in vivo skin tissue (FIGS. 10 and 11). The keratinocytes formed an epithelium-like layer, and enucleation, which is one of morphological indicators for keratinocyte differentiation, was observed (FIG. 11). In addition, the artificial skin at keratinocyte differentiation day 7 had a thickness (the distance from the surface of the epithelial tissue to the bottom surface of the bottom subcutaneous tissue) of about 4.3 mm. In view of the thickness, it was demonstrated that the thickness close to that of in vivo skin tissue was reproduced.

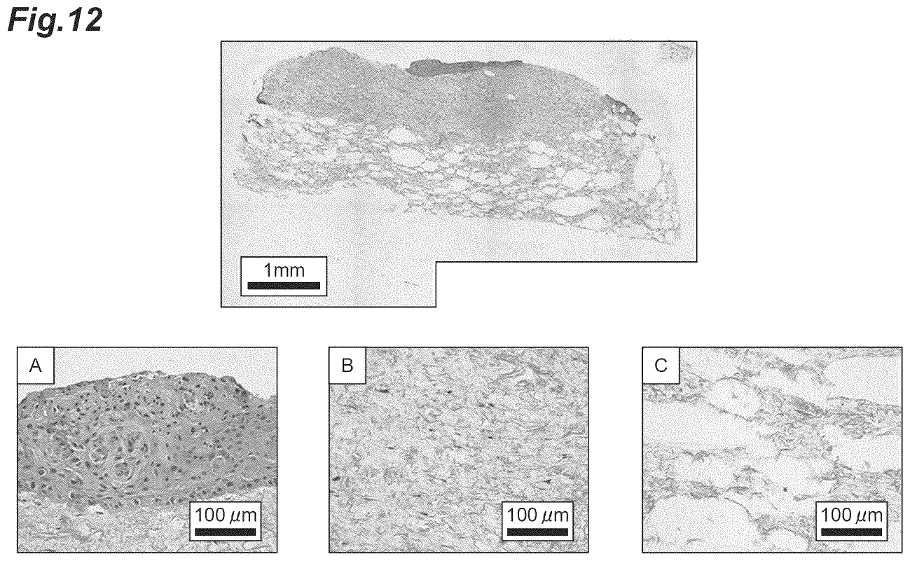

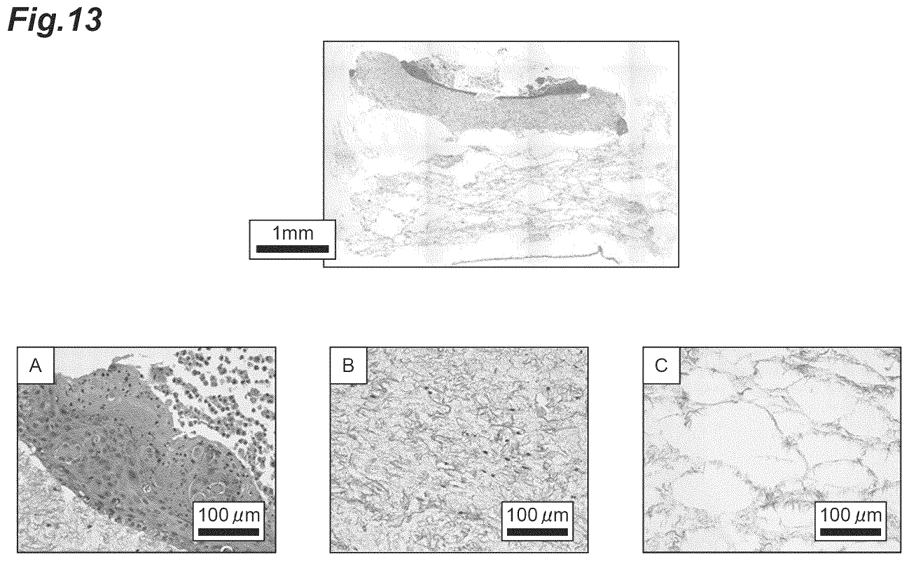

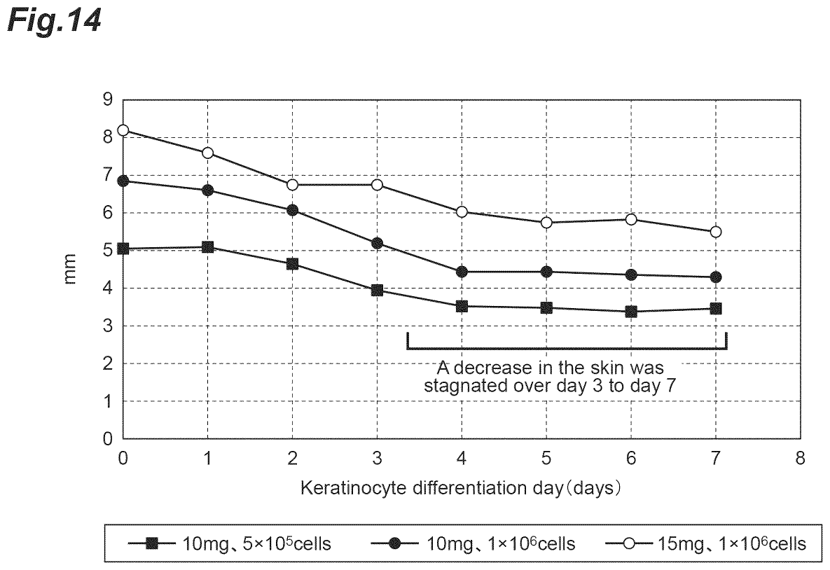

When the number of adipocytes used to produce an adipose tissue was changed from 1×10⁶ to 5×10⁵ and/or when the amount of fragmented collagen used was changed from 10 mg to 15 mg, substantially the same results as above were obtained (FIGS. 12 and 13). FIG. 14 shows how the thickness of artificial skin with a three-layer structure correlated to the keratinocyte differentiation time. It was indicated that a decrease in the thickness of the skin was slowed over keratinocyte differentiation day 3 to 7 and was stagnated.

### Example 4: Promotion of Adipogenesis in Adipose Stem Cells in Three-dimensional Tissue

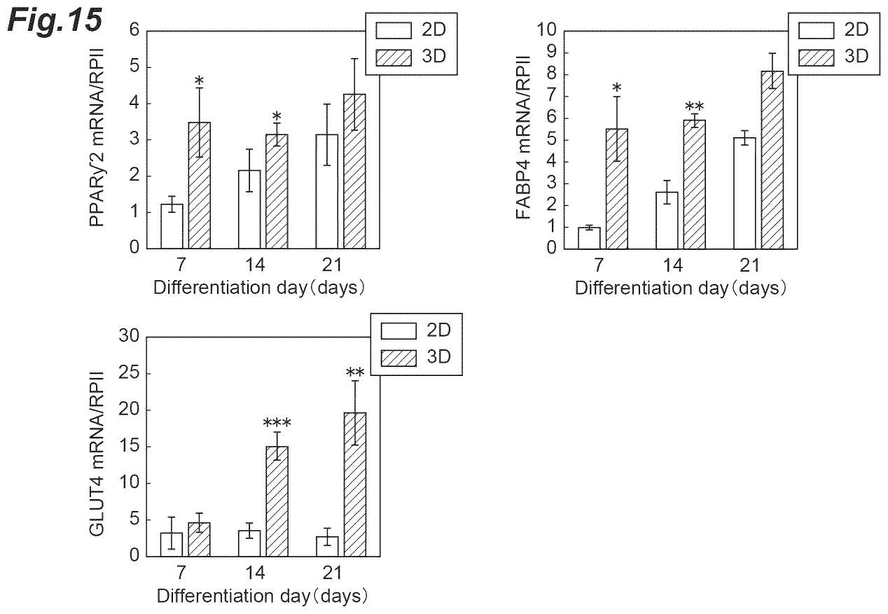

In a fragmented collagen-containing three-dimensional tissue produced, likewise in Example 2, using human adipose stem cells and an artificial adipose tissue produced by conventional two-dimensional culture, the levels of expression of adipogenesis genes were examined by real-time quantitative polymerase chain reaction (RT-qPCR) (FIG. 15).

A PureLink RNA Micro kit (manufactured by Invitrogen, Inc.) was used according to the kit protocol to extract total RNA from the above three-dimensional tissue or the artificial adipose tissue produced by conventional two-dimensional culture. The extracted RNA was quantified using Nanodrop (registered trademark) N1000 (manufactured by Thermo Fisher Scientific, Inc.). A High Capacity RNA-to-cDNA kit was used according to the kit protocol to convert 1 μg of the RNA to cDNA. In the PCR, 70 ng of the cDNA, 0.3 μM Forward and Reverse primers as well as iTaq (registered trademark) Universal SYBR Green Supermix (manufactured by Bio-Rad Laboratories, Inc.) were used for amplification. Table 1 shows each primer sequence used, the number of optimal amplification cycles in RT-PCR, and the temperature conditions. The cDNA synthesis and the RT-qPCR reaction were carried out using a StepOnePlus (registered trademark) real-time PCR system (manufactured by Thermo Fisher Scientific, Inc.).

TABLE 1

| | | SEQ | | Annealing |
|---|---|---|---|---|
| | | ID | The number | temperature |
| Gene | Primer sequence (5′ → 3′) | No. | of cycles | (° C.) |
| PPAR γ 2 | Forward: GCGATTCCTTCACTGATAC | 1 | 40 | 60 |
| | Reverse: GCATTATGAGACATCCCCAC | 2 | | |
| FABP4 | Forward: AACCTTAGATGGGGGTGTCC | 3 | 40 | 57 |
| (aP2) | Reverse: ATGCGAACTTCAGTCCAGGT | 4 | | |
| GLUT-4 | Forward: TTCCAACAGATAGGCTCCGAAG | 5 | 45 | 60 |
| | Reverse: AAGCACCGCAGAGAACACAG | 6 | | |
| RPII | Forward: CTTCACGGTGCTGGGCATT | 7 | 40 | 60 |
| | Reverse: GTGCGGCTGCTTCCATAA | 8 | | |

During adipogenesis, initial early adipogenesis genes (e.g., FABP4, PPARγ2) are expressed when preadipocytes become premature adipocytes. Then, while lipids accumulate in fat vesicles, late genes (e.g., GLUT4) are expressed. In this way, the growth stage is switched to the differentiation stage. The levels of expression of the early and late markers were found to gradually increase over the culturing period. However, a difference in the levels of expression was observed between the three-dimensional tissue and the artificial adipose tissue produced by two-dimensional culture. The levels of mRNA in the fragmented collagen-containing three-dimensional tissue tended to be higher than those of the artificial adipose tissue produced by two-dimensional culture. The level of expression of the early gene FABP4 at day 7 had a maximum 5.5-fold difference and the level of expression of the late gene GLUT4 at day 21 had a 8.3-fold difference (FIG. 15).

In the artificial adipose tissue produced by two-dimensional culture, the level of expression of the PPARγ2 gene has a continuous increase. By contrast, in the three-dimensional tissue, the expression profile in which the level increased slightly from day 7 to day 21 was seen, but the level was almost constant. In the three-dimensional tissue, PPAR12 was already at a constant expression stage, and adipogenesis seemed to have progressed more. In the three-dimensional tissue, the level of another early marker FABP4 stayed substantially the same from day 7 to day 21. By contrast, in the artificial adipose tissue produced by conventional two-dimensional culture, the level of expression was found to increase from day 7 to day 21. In the three-dimensional tissue, the level of expression of a late marker insulin-stimulated GLUT-4 rapidly increased from day 14. By contrast, in the artificial adipose tissue produced by two-dimensional culture, it was observed that the level of expression of GLUT-4 was very weak. These results have demonstrated that the above three-dimensional tissue has the gene expression levels reflecting differentiation states better than those of the artificial adipose tissue produced by two-dimensional culture.

### Example 5: Characterization of Fiber Diameter and Pore Size in Various Scaffold Materials

50 mg of lyophilized, pepsin-treated type I collagen from porcine skin (NH Foods Ltd.) was dispersed in 5.0 mL of 10×Phosphate-Buffered Saline (PBS, pH 7.4). The dispersion was then homogenized for 6 minutes using a homogenizer (VIOLAMO) to obtain a suspension of fragmented collagen components (Collagen Microfiber; CMF). For the preparation of collagen nanofiber (CNF), the suspension of fragmented collagen components (CMF) was stored at 4° C. for one day after being obtained. Matrigel (Corning Inc.) was prepared according to the manufacturer's instructions.

Their structures were observed using a Scanning Electron Microscope (SEM). Based on the obtained SEM images, the fiber diameter, pore area, and pore size were calculated using ImageJ software. The measurements were conducted for 100 samples (n=100) for each parameter.

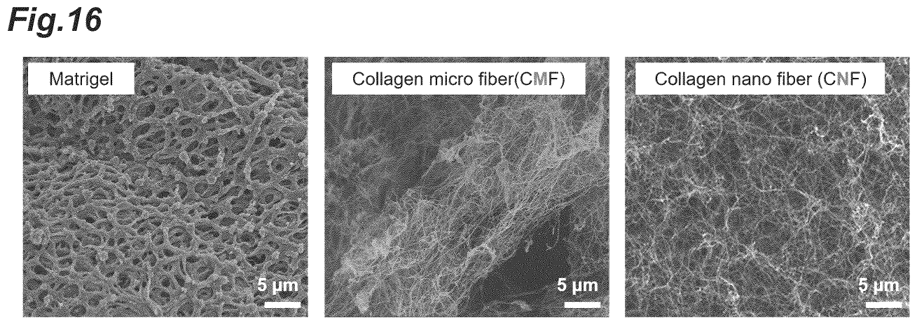

As shown in FIGS. 16, distinct structural differences were observed among the materials. Matrigel exhibited a fiber diameter of 0.79±0.38 μm, a pore area of 5.5±4.2 μm², and a pore size of 2.6±2.2 μm. CMF exhibited a fiber diameter of 10±9.1 μm, a pore area of 79±110 μm² and a pore size of 10.0±11.8 μm. CNF exhibited a fiber diameter of 0.078±0.047 μm, a pore area of 0.28±0.25 μm² and a pore size of 0.59=0.52 μm.

### Example 6: Detailed Structural Analysis of CMF and CNF

The CMF and CNF dispersions prepared in Example 5 were observed using phase-contrast microscopy and Transmission Electron Microscopy (TEM). The fiber diameters were measured from the obtained images using image analysis software (ImageJ).

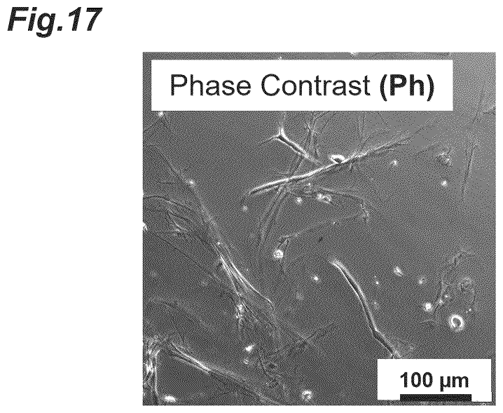

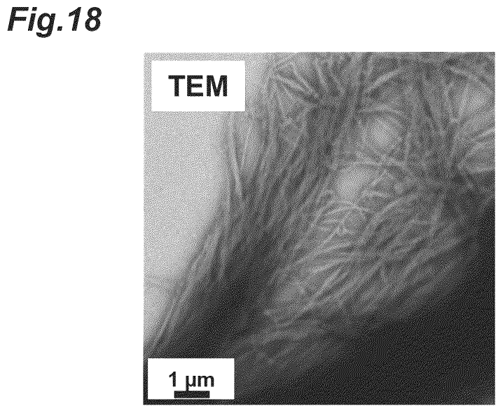

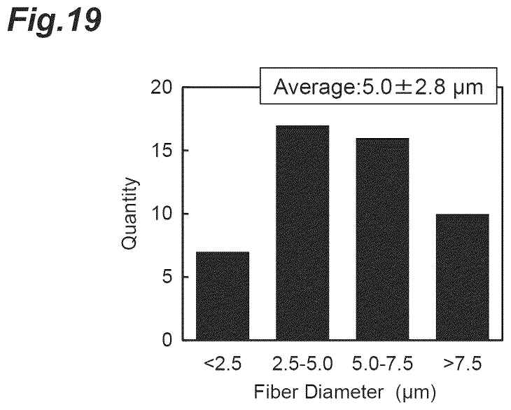

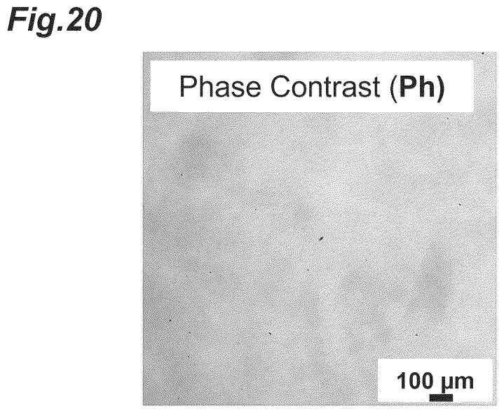

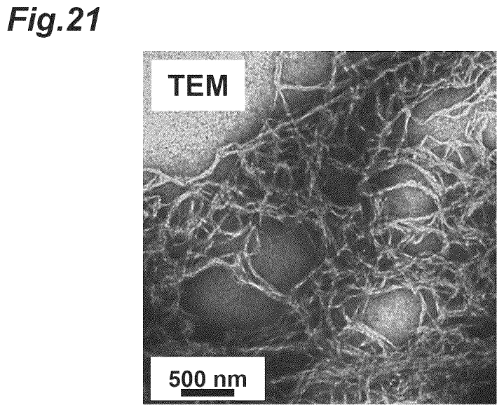

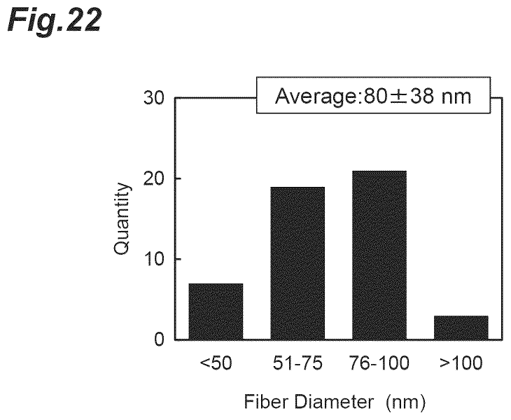

FIG. 17 is an image of CMF observed by phase-contrast microscopy, and FIG. 18 is an image of CMF observed by TEM. As shown in FIG. 19, the average fiber diameter of CMF was 5.0±2.8 μm. FIG. 20 is an image of CNF observed by phase-contrast microscopy, and FIG. 21 is an image of CNF observed by TEM. As shown in FIG. 22, the average fiber diameter of CNF was 80±38 nm.

### Example 7: Viscoelastic Analysis of CMF and CNF Gels

CMF and CNF (prepared in Example 5) dispersions were prepared at a concentration of 1.0 wt % using phosphate-buffered saline (PBS). The viscoelasticity, specifically the Storage Modulus (G′) and Loss Modulus (G″), was measured using a rheometer (Anton Paar Japan K.K., MCR302). The measurement was conducted in a frequency sweep mode ranging from 0.1 Hz to 1 Hz with a shear stress of 10 Pa. The measurements for the CMF sol were performed at room temperature, while those for the CNF sol were performed at 4° C. To prepare the gel samples, the CMF and CNF sols were incubated at 37° C. for 3 hours. The formation of gels was confirmed visually before conducting the rheological measurements at 37° C.

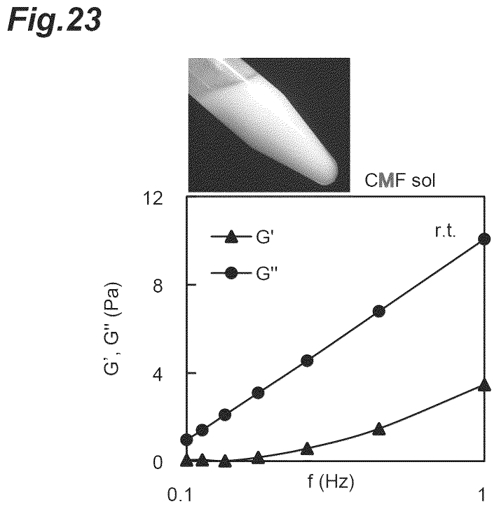

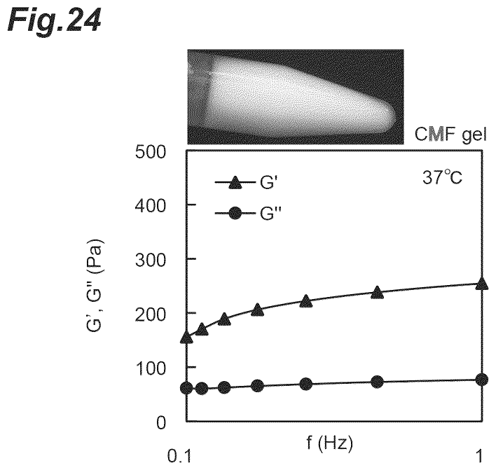

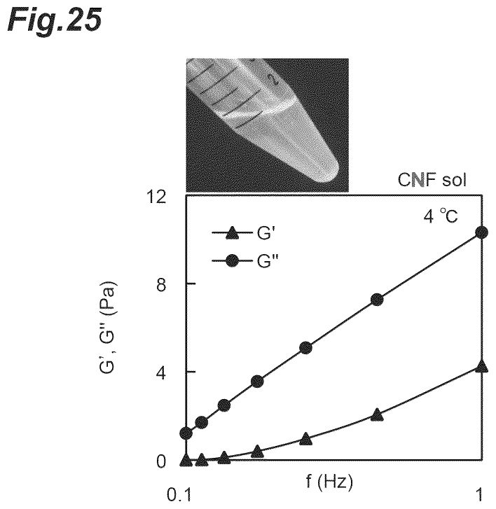

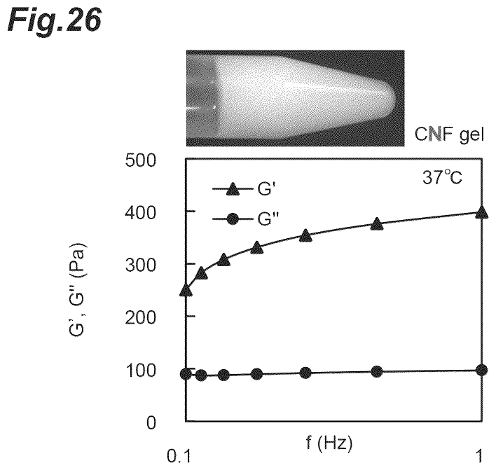

As shown in FIGS. 23 to 26, the rheological measurements revealed distinct behaviors for the sol and gel states. For the CMF sol at room temperature (FIG. 23) and the CNF sol at 4° C. (FIG. 25), both G′ and G″ values were low, indicating a liquid-like state. After incubation at 37° C. for 3 hours, both CMF and CNF dispersions formed white, turbid gels. The rheological data at 37° C., shown in FIG. 24 for the CMF gel and FIG. 26 for the CNF gel, showed that the Storage Modulus (G′) was significantly higher than the Loss Modulus (G″) for both materials, which is characteristic of a gel state. Furthermore, the CNF gel (FIG. 26) exhibited a higher G′ of approximately 400 Pa at 1 Hz compared to the CMF gel (FIG. 24), which showed a G′ of approximately 250 Pa at 1 Hz.

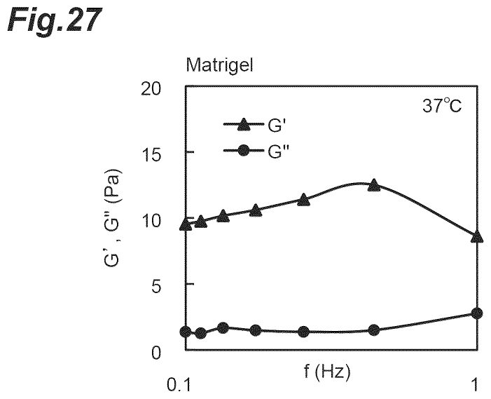

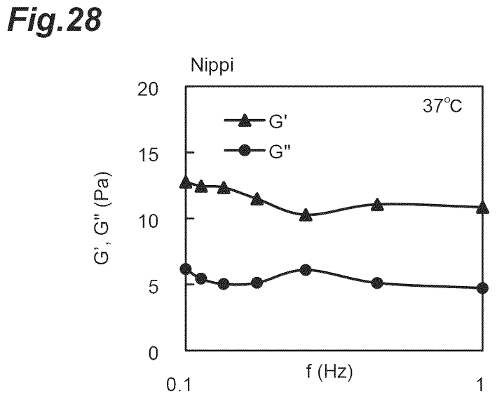

The viscoelasticity of CMF and CNF gels were compared with widely used commercially available scaffold materials: Matrigel and Nippi collagen. Matrigel (basement membrane matrix) and Nippi collagen (type I collagen solution) were neutralized and prepared according to their respective manufacturer's instructions to form gels at 37° C. The viscoelastic properties were measured at 37° C. under the same conditions as described above. As shown in FIG. 27 (Matrigel) and FIG. 28 (Nippi collagen), both commercial materials exhibited significantly lower viscoelastic moduli compared to the CMF and CNF gels. Specifically, the Storage Modulus (G′) at 1 Hz was approximately 10 to 15 Pa for both Matrigel and Nippi collagen. In contrast, as described above, the G′ values for CMF and CNF gels were approximately 250 Pa and 400 Pa, respectively. These results demonstrate that the CMF and CNF gels possess mechanical strengths that are more than 10 to 20 times higher than those of conventional collagen-based or basement membrane-based hydrogels.

### Example 8: Measurement of Elastic Modulus of CMF and CNF Gels

CMF and CNF dispersions were prepared at concentrations of 0.2, 0.5, 1.0, 2.0, 3.0, and 4.0 wt % using PBS. CMF and CNF were prepared by the same method as in Example 5. As a comparative control, Matrigel was also prepared according to the manufacturer's instructions. All dispersions (CMF, CNF, and Matrigel) were incubated at 37° C. to form gels. The elastic modulus was measured using a compression tester (EZ-Test, Shimadzu Corporation). The elastic modulus (E) was calculated based on the stress-strain curve using the following formula: E (mN/mm²=kPa)={H (mm)/C (mm²)}×S (mN/mm) where H is the gel height (mm), C is the contact area (mm²), and S is the slope of the stress-strain curve at a strain of 50% or less (mN/mm).

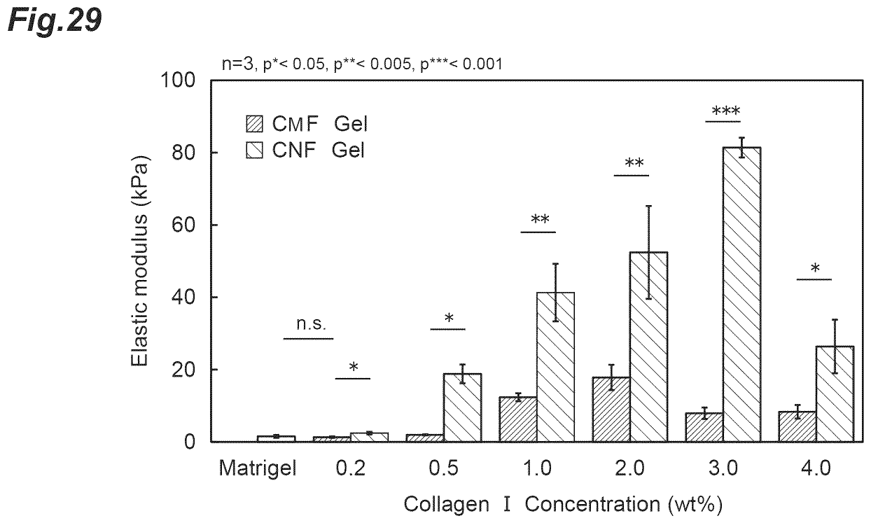

As shown in FIG. 29, the elastic modulus of both CMF and CNF gels generally increased with collagen concentration. The elastic modulus of CMF and CNF peaked at a concentration of 3.0 wt %. Specifically, the 3.0 wt % CNF gel achieved an elastic modulus of approximately 80 kPa.

### Example 9: Quantitative Analysis of Hydrophobic Environments within Gels using a Hydrophobic Probe

To prepare samples for fluorescence intensity measurement, fragmented extracellular matrix (ECM) components (CMF and CNF) were added to 1× Phosphate-Buffered Saline (PBS) to a final concentration of 0.5 wt % and stored in a refrigerator for 4 days to allow dissolution, resulting in a “fragmented ECM solution.” CMF and CNF were prepared by the same method as in Example 5. Separately, Sodium 8-anilino-1-naphthalenesulfonate (ANS-Na) was dissolved in 1×PBS to a final concentration of 20 μM to prepare an “aqueous ANS solution.” Subsequently, 200 μL of the aqueous ANS solution was added to 500 μL of the fragmented ECM solution, and the mixture was allowed to react for one hour at room temperature (25° C.) protected from light. The fluorescence intensity of the resulting reaction mixture was measured using a fluorospectrometer (e.g., NanoDrop™ 3300 Fluorospectrometer, Thermo Fisher Scientific) with an excitation wavelength of 380 nm.

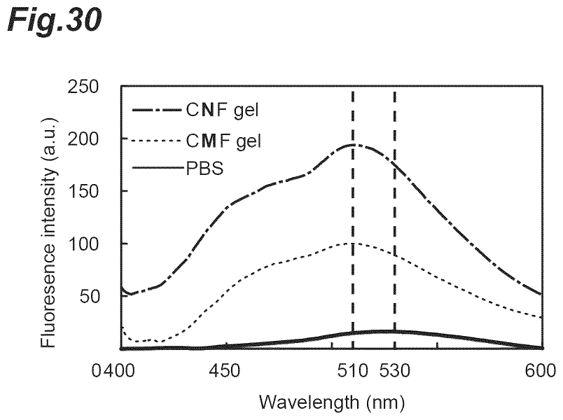

As shown in FIG. 30, the fluorescence spectra revealed significant differences in the hydrophobic environments provided by the materials. The PBS control showed negligible fluorescence. The CMF gel exhibited a moderate fluorescence intensity. In contrast, the CNF gel exhibited a significantly higher fluorescence intensity, which was approximately twice that of the CMF gel. Since ANS exhibits increased fluorescence in hydrophobic environments, this indicates that the CNF gel possesses a larger amount of hydrophobic domains compared to the CMF gel. Furthermore, a shift in the fluorescence peak wavelength was observed. While the CMF gel showed a peak around 530 nm, the CNF gel showed a peak shift to a shorter wavelength of approximately 510 nm. The combination of increased intensity and the shift suggests that the finer nanofiber network of CNF creates a more extensive and specific hydrophobic environment, which is distinct from the environment in CMF microfibers.

### Example 10: Visualization of Hydrophobic Domains in Gels using ANS-Na

CMF and CNF dispersions were prepared at a concentration of 0.5 wt % using PBS. CMF and CNF were prepared by the same method as in Example 5. As a control, a Nippi collagen gel was prepared at a concentration of 0.1 wt %. ANS-Na was added to each dispersion to achieve a final concentration of 1 mM. The mixtures were incubated at 37° C. to induce gelation. The prepared gels were observed using a fluorescence microscope. The imaging conditions were set with an excitation wavelength of 405 nm, and the fluorescence emission was collected in the range of 500 nm to 550 nm. The fluorescence intensity per unit area was quantified from the obtained images using ImageJ software.

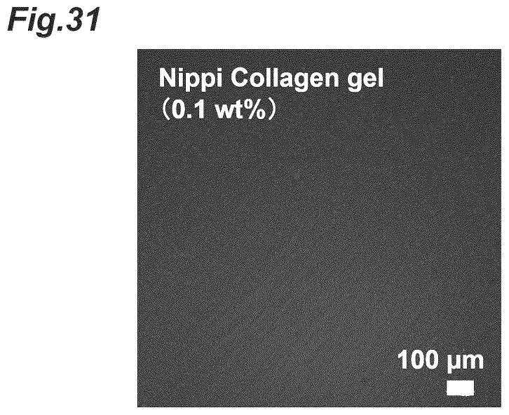

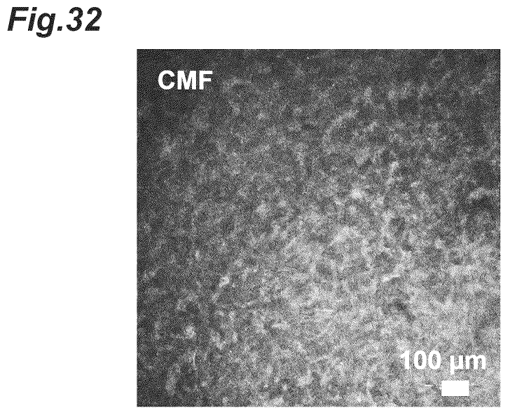

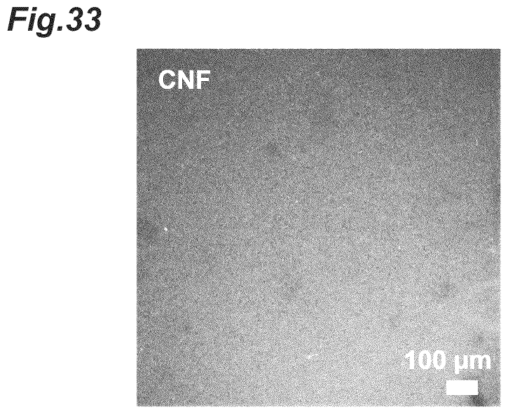

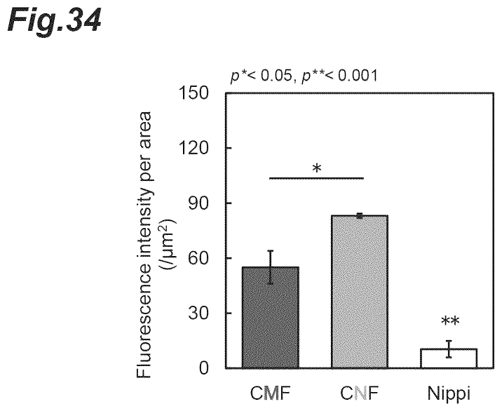

As shown in FIGS. 31 to 34, the fluorescence images revealed distinct differences in the microstructure and hydrophobicity among the gels. FIG. 31 shows the result for the Nippi collagen gel (0.1 wt %), which appeared dark with very low fluorescence intensity, indicating a lack of significant hydrophobic domains detectable by ANS-Na under these conditions. FIG. 32 shows the result for the CMF gel (0.5 wt %), which exhibited a fibrous network structure with moderate fluorescence intensity. FIG. 33 shows the result for the CNF gel (0.5 wt %), which displayed a dense and uniform fluorescence with significantly higher intensity compared to CMF and Nippi collagen. FIG. 34 shows the quantitative analysis results. It was confirmed that the fluorescence intensity per area of the CNF gel was significantly higher than that of the CMF gel (p<0.05) and the Nippi collagen gel (p<0.001).

### Example 11: Requirements for the CNF Sol-Gel Transition

The conditions required for the reversible sol-gel transition of the CNF dispersion were investigated. Specifically, the effects of salt concentration, collagen concentration, and pH on the gelation behavior were evaluated.

### 1. Effect of Salt Concentration

To investigate the effect of salt concentration on the sol-gel transition, Collagen Nano Fibers (CNF) were first prepared using a high-salt method. Specifically, 50 mg of lyophilized, pepsin-treated type I collagen from porcine skin (NH Foods Ltd.) was dispersed in 5.0 mL of 10×Phosphate-Buffered Saline (10×PBS, pH 7.4) supplemented with 137 mM NaCl. This mixture was homogenized for 6 minutes using a homogenizer (VIOLAMO) to yield a suspension of fragmented collagen components (CMF). The suspension was washed, centrifuged, and subsequently stored at 4° C. for one day to obtain CNF. Dispersions at a concentration of 1.0 wt % were prepared under two distinct salt conditions: 0 mM NaCl, prepared separately without the addition of NaCl, and 137 mM NaCl. The optical transmittance of these samples was measured using a spectrophotometer (V-670, JASCO Corporation) at a wavelength of 500 nm. The temperature of the samples was controlled by gradually increasing or decreasing the temperature at a rate of 1° C./min while monitoring the transmittance.

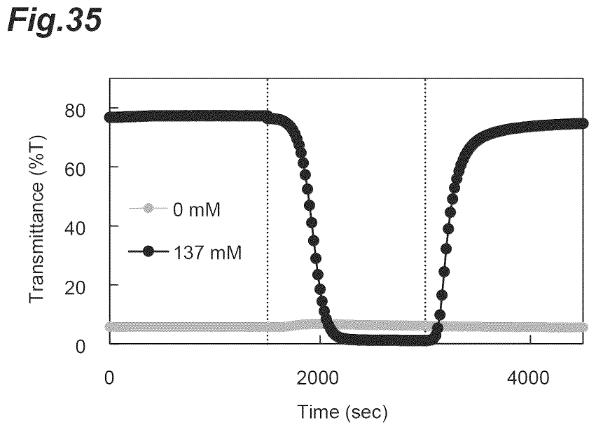

As shown in FIG. 35, the sample containing 137 mM NaCl exhibited a high transmittance of approximately 80% in the initial state (at 4° C.), indicating a clear sol. Upon heating to 37° C., the transmittance decreased rapidly to approximately 0%, signifying the formation of a turbid gel. Furthermore, upon cooling back to 4° C., the transmittance recovered to approximately 80%, indicating a return to the sol state. In contrast, the sample containing 0 mM NaCl showed low transmittance throughout the measurement and did not exhibit the reversible sol-gel transition behavior. These results confirm that a physiological salt concentration, such as 137 mM, is essential for the thermally reversible sol-gel transition of the CNF.

### 2. Effect of Collagen Concentration

To determine the concentration threshold for gelation, CNF dispersions were prepared at concentrations of 0.1 wt % and 1.0 wt % in 1×PBS (pH 7.4). The samples were placed in test tubes and incubated at 37° C. for 3 hours, after which their state (sol or gel) was evaluated by visual inspection.

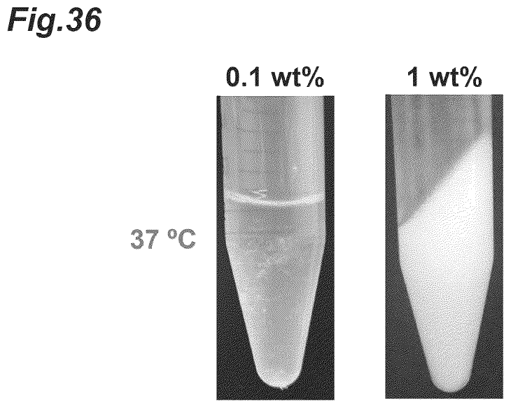

As shown in FIG. 36, the 0.1 wt % sample remained a clear, flowing liquid (sol) even at 37° C. On the other hand, the 1.0 wt % sample formed a white, turbid solid (gel) that did not flow when the tube was inverted. This result indicates that a certain threshold of collagen concentration (higher than 0.1 wt %, e.g., 0.5 wt % or more) is required to form a stable gel network at 37° C.

### 3. Effect of pH

To evaluate the effect of pH, a CNF dispersion was prepared under acidic conditions using 5 mM acetic acid (pH approx. 3-4) instead of neutral PBS. The sample was subjected to a temperature cycle consisting of an initial state, heating to 37° C., cooling to 4° C., and reheating to 37° C., with the state being observed visually at each stage.

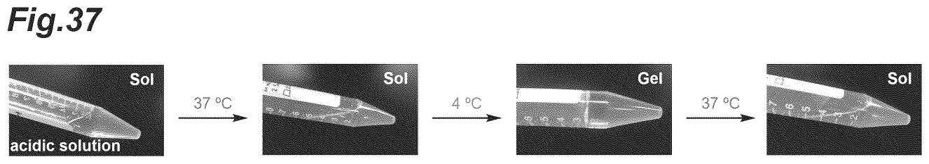

As shown in FIG. 37, initially, the sample was a solution (Sol). At 37° C., it remained a solution and did not form the white turbid gel. Upon cooling to 4° C., the sample formed a gel, a behavior characteristic of gelatin or denatured collagen. Upon reheating to 37° C., the sample returned to a solution. This demonstrates that under acidic conditions, the desired “sol at low temperature/gel at body temperature” transition does not occur. Therefore, neutral conditions are essential for the specific sol-gel transition mechanism of the present invention.

### Example 12: Investigation of Sol-Gel Transition

A type I collagen acidic solution (3 mg/mL in 5 mM acetic acid, Nippi, Inc.) derived from porcine skin was used as the starting material. The solution was neutralized by adding PBS and NaOH and heated at 37° C. for 1 hour to induce gelation. Subsequently, the gel was freeze-dried at −80° C. for 7 days, and the dried collagen was physically disaggregated using a homogenizer. This cycle of freeze-drying and physical disaggregation was performed twice. Finally, the obtained CMF was dispersed in MilliQ water and PBS at concentrations of 0, 0.1, 0.2, 0.5, 0.8, and 1.0 wt %, and stored at 4° C. overnight.

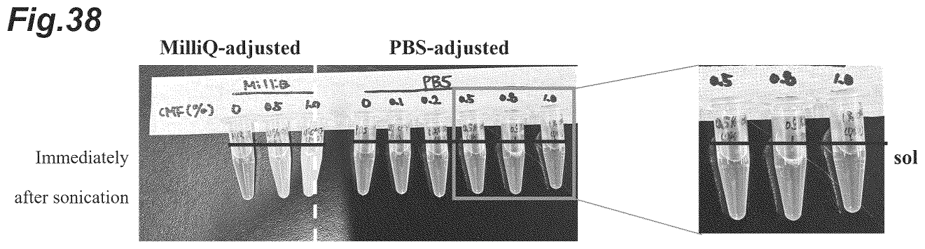

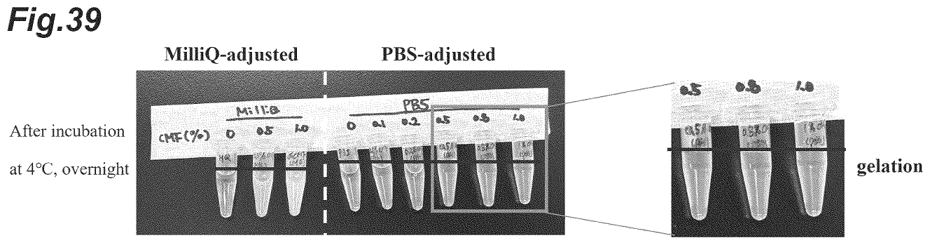

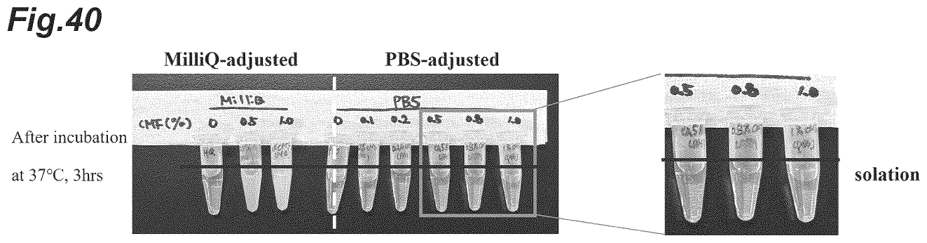

The state of the CMF dispersions was observed after storage at 4° C. and after incubation at 37° C. for 3 hours. As shown in FIG. 38-40, a reverse sol-gel transition, from a gel state at 4° C. to a sol state at 37° C., was observed specifically for the samples prepared with PBS at concentrations of 0.5 wt % and higher. These specific samples formed a gel (white turbid solid) after overnight storage at 4° C. (FIG. 39) and subsequently underwent solation upon heating to 37° C. (FIG. 40). In contrast, this reversible transition was not observed in the other samples. Both the samples prepared with Milli-Q water and the PBS samples with lower concentrations (0.1 and 0.2 wt %) appeared to remain in a gel-like state throughout the temperature cycle, failing to undergo solation at 37° C.

### Example 13: Investigation of Salt Concentration Range for Sol-Gel Transition

To determine the precise range of salt concentrations that allow for the reversible sol-gel transition, an experiment was conducted using samples with accurately adjusted salt concentrations. The starting material was NMP Collagen PS (Nippi, Inc.), a powdered Type I/III collagen derived from porcine skin. The collagen powder was dispersed in 10×PBS and physically disaggregated using a homogenizer, and the resulting suspension was centrifuged at 9500 rpm for 5 minutes to obtain a collagen pellet. To eliminate the influence of residual salt and to ensure accurate salt concentrations for evaluation, the obtained collagen pellet was dialyzed against pure water and then freeze-dried to obtain salt-free collagen sponges. This salt-free collagen was redissolved in various solvents to prepare 1.0 wt % dispersions, including MilliQ water and PBS at various concentrations ranging from 0.1× to 10×PBS (specifically, 0.1×, 0.2×, 0.5×, 1×, 5×, and 10×PBS). The reconstituted dispersions were stored at 4° C. overnight to promote the formation of CNF. Before observation, the samples were centrifuged at 4500 rpm for 3 minutes to remove any large aggregates. Finally, the samples were subjected to a temperature cycle (4° C.->37° C.->4° C.), and their sol-gel transition behavior was observed visually.

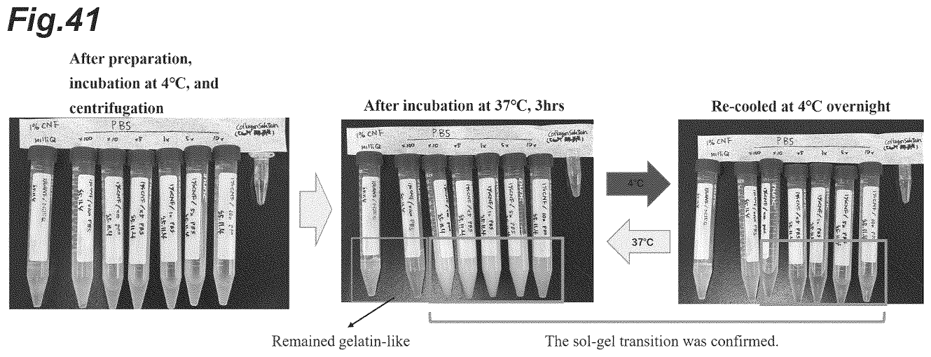

As shown in FIG. 41, the behavior of the samples depended on the salt concentration. The sample prepared with MilliQ water (and ×100 diluted PBS) appeared turbid or gelatin-like even at 4° C. and did not exhibit the clear sol-gel transition characteristic of CNF. In contrast, samples prepared with PBS concentrations ranging from 0.1× to 10× exhibited the desired reversible behavior. Specifically, they were clear, flowing liquids (Sol) at 4° C. and formed white, turbid solids (Gel) at 37° C.

### Example 14: Quantitative Viscoelastic Analysis at Various Salt Concentrations

The salt-free collagen prepared in Example 13 was redissolved in MilliQ water and PBS at various concentrations (×100, ×10, ×5, 1×, 5×, and 10×) to a final collagen concentration of 1.0 wt %. The viscoelastic properties of each sample were measured using a rheometer (Anton Paar Japan K.K., MCR302). A frequency sweep test was conducted to measure the frequency dependence of the storage modulus (G′) and loss modulus (G″). The strain rate was fixed at a value confirmed to be within the linear viscoelastic region based on a prior strain sweep test. The measurement frequency range was from 1.0 Hz to 10.0 Hz, with over 20 data points set at logarithmic or linear intervals within this range, yielding G′ [Pa] and G″ [Pa] at each frequency.

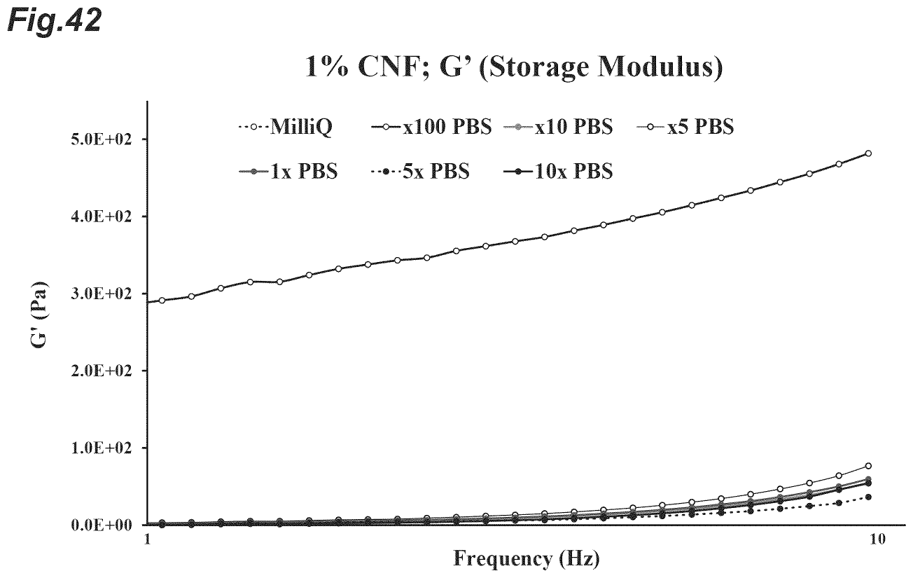

As shown in FIGS. 42 (G′) and 43 (G″), the viscoelastic properties of the suspensions were evaluated based on the obtained G′ and G″ data. The samples prepared with MilliQ water and ×100 diluted PBS maintained a gel-like state. Specifically, the ×100 diluted PBS sample showed G′ values significantly higher than G″ values across the frequency range, which is characteristic of a solid-like gel (note: the data for MilliQ water completely overlaps with the data for ×100 diluted PBS and is therefore obscured in the graph). In contrast, for the samples prepared with PBS concentrations of ×10 diluted or higher (×10, ×5, 1×, 5×, and 10×PBS), it was confirmed that G″ was greater than G′ over the measurement frequency range, and both G′ and G″ were below 20 Pa.

This indicates that these suspensions maintained a stable, liquid-like sol state.

1. An artificial adipose tissue comprising:
a three-dimensional tissue comprising a population of cells including adipocytes; and
a naturally derived fragmented extracellular matrix having an average length from 100 nm to 400 μm and being insoluble in water,
wherein the fragmented extracellular matrix to be in contact with the cells with at least a part of the cells adhered to the fragmented extracellular matrix in form of the three-dimensional tissue,
wherein the cells and the fragmented extracellular matrix are uniformly distributed in the artificial adipose tissue in form of the three-dimensional tissue, and
the adipocytes have an average size of lipid droplets from 20 μm to 180 μm.

2. The artificial adipose tissue according to claim 1,
wherein the fragmented extracellular matrix has undergone gelation from a sol state at 35.5° C.±2° C. and undergoes solation from a gel state at 4.5° C.±2° C.

3. The artificial adipose tissue according to claim 1,
wherein the fluorescence intensity of the fragmented extracellular matrix at a wavelength of 510 nm is 50 a.u. or more and 300 a.u. or less, when measured with a spectrofluorometer using a fluorescent hydrophobic probe upon excitation at 380 nm.

4. A method for producing an artificial adipose tissue, comprising:
(1) contacting a population of cells including an adipocyte with a fragmented extracellular matrix in an aqueous medium; and
(2) culturing the cells contacted with the fragmented extracellular matrix.

5. The method according to claim 4,
wherein the pore area of the fragmented extracellular matrix is 10 μm² or more and 500 μm² or less.

6. The method according to claim 4,
wherein the pore size of the fragmented extracellular matrix is 1 μm or more and 50 μm or less.

7. The method according to claim 4, further including the steps of subjecting
the fragmented extracellular matrix to gelation from a sol state at 35.5° C.±2° C. and to solation from a gel state at 4.5° C.±2° C.

8. The method according to claim 4, further including the steps of subjecting
the fragmented extracellular matrix through one or more cycles of freezing and thawing.

9. The method according to claim 8,
wherein the pore area of the fragmented extracellular matrix is 0.01 μm² or more and 1 μm² or less.

10. The method according to claim 8,
wherein the pore size of the fragmented extracellular matrix is 0.01 μm or more and 10 μm or less.

11. The method according to claim 4,
wherein the NaCl concentration of the aqueous medium in step (1) is 13.0 mM or more and 1400 mM or less.

12. The method according to claim 4,
wherein the fluorescence intensity of the fragmented extracellular matrix at a wavelength of 510 nm is 50 a.u. or more and 300 a.u. or less, when measured with a spectrofluorometer using a fluorescent hydrophobic probe upon excitation at 380 nm.
